(19) United States
(12) Patent Application Publication
(10) Pub. No.: US 2024/0393310 A1
(43) Pub. Date: Nov. 28, 2024

(54) BIOMARKER REFLECTANCE SIGNATURES FOR DISEASE DETECTION AND CLASSIFICATION

(71) Applicant: University of Florida Research Foundation, Incorporated, Gainesville, FL (US)

(72) Inventors: Ioannis AMPATZIDIS, Gainesville, FL (US); Jeanette HARIHARAN, Gainesville, FL (US)

(21) Appl. No.: 18/693,658

(22) PCT Filed: Oct. 4, 2022
(86) PCT No.: PCT/US2022/077495

§ 371 Date: Mar. 20, 2024

Related U.S. Application Data
(60) Provisional application No. 63/252,755, filed on Oct. 6, 2021.

(51) Int. Cl. G01N 33/00; G01N 21/17; G01N 21/31; G01N 21/84; G16B 40/10

(52) U.S. Cl. CPC G01N 33/0098; G01N 21/31; G16B 40/10; G01N 2021/1797; G01N 2021/8466

(57) ABSTRACT

Various embodiments of the present disclosure provide systems and methods for detecting and classifying a disease state of a plant based at least in part on generating a reflectance signature for the plant. The reflectance signature is generated using a reduced set of orthogonal signal components determined from the Karhunen-Loeve Expansion. Various embodiments enable the classification of different disease states (e.g., healthy, asymptomatic, early stage, late stage) of a particular plant disease and the classification of disease states of different plant diseases, such as the differentiation between plants infected with a first plant disease from plants affected with another plant condition. The reflectance signature is generated using reduced-spectrum frequency data extracted or processed from reflectance signal data obtained from the plant. One or more signal components of the reflectance signal data that accurately describe at least a threshold amount of variance or energy of the reflectance signal data are selected for generation of the reflectance signature.

### REFERENCE TO RELATED APPLICATIONS

This application claims priority to and the benefit of U.S. Provisional Application. No. 63/252,755, filed on Oct. 6, 2021, the entire contents of which are incorporated herein by reference.

### GOVERNMENT SUPPORT

This invention was made whole or in part through a subrecipient grant awarded by the United States Department of Agriculture, Agricultural Marketing Services through the Florida Department of Agricultural and Consumer Services.

### TECHNOLOGICAL FIELD

Embodiments of the present disclosure generally relate to systems and methods for programmatically detecting and classifying a disease state of a plant.

### BACKGROUND

Detecting plant diseases and stress factors in the early stages of disease development is essential for effective crop production management.

One such plant disease is citrus canker (CC), which is a serious bacterial disease of citrus worldwide caused by Xanthomonas citri subsp. citri (Xcc; syn. X. axonopodis pv. citri). CC is often found in Florida's Sugar Belle mandarin (Citrus reticulata) trees. The bacterium is dispersed by wind and rain and prefers humid-wet climates. The Florida citrus industry has suffered serious consequences over the last decades, and methods for early detection of citrus canker have become critical. On severely infected trees, the pathogen can cause severe fruit blemishes and premature leaf and fruit drop, resulting in significant economic impacts. The symptoms include raised necrotic lesions with yellow halos on leaves, twigs, and fruits. The control of citrus canker has been a challenge because the pathogen is difficult to eradicate due to its wide host range, a long latent period in asymptomatic trees, and high inoculum potential from rain and wind-blown lesions. In dry seasons, a plant may look healthy, but the bacterial growth stages may take a few months to show symptoms.

Another example of a lethal plant disease is laurel wilt (Lw), which attacks avocado (Persea americana) trees in Florida. The economic impact of Lw disease on the Florida avocado industry was estimated to be up to $54 million in the absence of effective control measures. Laurel wilt is caused by Raffaelea lauricola, which is a symbiont fungus of its insect vector, the Asian ambrosia beetle Xyleborus glabratus Eichhoff. R. lauricola fungus infects plant vasculature and blocks the flow of water and nutrition to the leaves. In the first days of infection (early disease development stage), the symptoms appear as yellowish leaves, and in the late stages, the leaves turn to a brownish color with necrotic and curly areas. These symptoms are similar to nutritional (e.g., iron (Fe) and nitrogen (N)) deficiencies in avocado trees, and some resemble those caused by diseases such as Phytophthora root rot and Verticillium wilt and other abiotic factors such as lightning and frost damage.

Laboratory analysis of plant samples for disease detection is invasive, costly, time-consuming, and labor-intensive. For that reason, several disease detection methods have been developed utilizing advanced and sophisticated spectral data analysis approaches without harming the plant. Common preprocessing and analysis techniques include normalization and derivative spectra enhancement using various methods, such as finite differencing, complex step derivative, and derivative spectral shape equation. Wavelet Transform has been used and compared to derivative spectra enhancement and has shown to be very successful in spectral regions of interest; it is becoming more commonly used as an alternative to spectral derivative methods. Interpolating polynomials are also used to smooth the (spectral) data and better represent enhanced spectra. Multivariate analysis can be used to gain a better understanding of spectral variance between diseased and healthy reflectance properties.

More recently, ground and unmanned aerial vehicles (UAVs) with hyperspectral camera payloads have been used to collect data for improving agricultural production.

Along with this data collection method, deep and transfer learning artificial intelligence (AI) applications have been developed for biotic and abiotic stress detection in plants. These techniques required a high-quality training dataset to accurately develop the prediction models. All machine learning algorithms require some data preprocessing, other segmentation methods, and feature extraction to obtain prediction models for disease detection adequately.

### BRIEF SUMMARY

While known methods for the detection and classification of plant diseases are becoming more robust and refined, there has not been much interest in trying to understand the underlying frequency components that characterize plant signatures. Accordingly, there is a need for a method to define these signatures so that a database could be developed for healthy plant signatures and variants of those signatures that differentiate disease(s) in the species. Because of the high number of frequency components that represent the (spectral) data, a reduced spectrum based at least in part on the highest energy frequencies of variance would be desirable.

These highest energy frequencies can be determined by their eigenvalues associated with a multi-variate regression representation of the data. These modified spectrums can be incorporated into a model to reduce the data while maintaining the relevant characteristics that define each plant reflectance signature. Using multivariate analysis, it is possible to prove that specific variance patterns exist in a diseased plant that differs from the healthy plant, over a mean statistical population. Taking the eigenvector components and establishing a basis vector for the classification of healthy core species and the disease factor species, fundamental waveforms or basis signatures can then be computed to help identify diseases and establish baseline biomarkers for different diseases in different species of plants.

Accordingly, embodiments of the presently disclosed system and method concern determining optimized reflectance spectra (e.g., signatures) of plants, and how these spectra vary when various diseases and nutrient deficiencies are present. Plant reflectance data from hyperspectral imaging sensors is collected. A Karhunen-Loeve Expansion (KLE) of spectral reflectance data, taken from healthy and diseased plants (e.g., citrus and avocado species) is used to identify a basis set of functions that represents the distribution of the reflected signal energy by defining the highest absolute value of the eigenvectors responsible for the areas of greatest variation in the reflectance signal data of plants with different disease states and how these patterns are interrupted and changed by disease and malnutrition effects. By spectral decomposition, the eigenvalues are related to the KLE basis set and used to identify the KLE eigenvectors, which comprise the highest variation in the data. These components are interpreted as weighted variables that carry with them most of the information on the reflectance spectrum of the plant and are used to develop a series truncation process in the Fourier domain, resulting in a reduced dataset for spectral analysis. Based at least in part on this multivariate KLE analysis, an adapted frequency reconstruction is performed to convert the eigenvector information to a wave function. This reconstruction via KLE and frequency transformation form a signature identification process (e.g., generating a unique biomarker signature), as the frequency spectra are used as average signature reflectance patterns for plant identification, classification, and disease biomarkers. Spectral dictionaries or databases of healthy and diseased plant signatures for classification purposes can thus be developed.

Defining these spectral identification biomarkers or signatures allows for less invasive classification and disease diagnostics. Examining inherent biomarkers in plants also enables a deeper and more accurate analysis compared to visual analysis. Additionally, generating a database of healthy and disease species' reflectance signatures is very useful for plant identification, early disease diagnostics, disease specification, abiotic stress related factors' recognition, and general crop assessment.

Moreover, reduction of reflectance signal data to high-energy and high-variance frequency components that carry the most relevant information of the reflectance signal data enables improved efficiency in computational and processing resources, due to there being less data and fewer dimensions thereof to analyze.

In general, embodiments of the present disclosure provide methods, apparatuses, systems, computing devices, computing entities, and/or the like for detecting and classifying a disease state of a plant based at least in part on generating a reflectance signature for the plant. Various embodiments examine the underlying frequency components in reflectance signal data that characterize a plant and define signatures based at least in part on such frequency components. In various embodiments, these defined signatures are used to develop a database for healthy plant signatures, asymptomatic plant signatures, early disease stage plant signatures, late disease stage plant signatures, and/or various other variants that differentiate disease(s).

Example embodiments of the present disclosure can be applied to detect and classify disease states for different disease and for different plants. In one example, one or more stages of citrus canker can be classified in the Sugar Belle Mandarin plant. In another example, different nutritional deficiencies and diseases can be detected in avocado trees. In further examples, different diseases (e.g., target spot, bacterial spot) can be detected in tomatoes, powdery mildew can be detected and classified in squash, and downy mildew can be detected and classified in watermelon. It will be understood that these diseases, deficiencies, and plants are exemplary in nature, and that various embodiments of the present disclosure provide for detecting and classifying disease states of various diseases for various plants not limited by examples discussed herein. The present disclosure could extend to all crops, cover crops, biocrusts, herbaceous plants, woody plants, and could even be used for soil and other geological analysis.

In general, according to one aspect, the invention features a method for detecting diseases in plants. Reflectance signal data for a plant is received, in which a plurality of signal components is identified. Within this plurality of signal components, signal components having a variance satisfying a variance threshold are then selected, the variance being between particular signal components of reflectance signal data for a non-diseased plant and corresponding signal components of reflectance signal data for a diseased plant. Reduced-spectrum frequency data is generated based at least in part on the reflectance signal data and the selected signal components. A reflectance signature for the plant is then generated based at least in part on the reduced-spectrum frequency data, at which point a disease state of the plant is then determined based at least in part on the reflectance signature.

In embodiments, a signature database configured to describe one or more average reflectance signatures for a plant species is generated, with each of the average reflectance signatures being associated with a particular disease state of the plant species. These average reflectance signatures include healthy plant signatures, asymptomatic plant signatures, early disease stage plant signatures, late disease stage plant signatures, and/or nutrient-deficient plant signatures, to name a few examples.

In examples, the reflectance signature comprises a truncated frequency series associated with the reflectance signal data, one or more power spectral density magnitudes associated with the selected signal components, and/or one or more phases associated with the selected signal components.

The selected signal components are identified based at least in part on using the Karhunen-Loeve Expansion as a kernel function for the Mercer's Theorem, for example.

The disease state of the plant is determined based at least in part on providing the reflectance signature to one or more machine learning models configured to predict a disease state of a plant when provided with an input reflectance signature. These machine learning models comprise a clustering model, a bivariance correlation model, and/or a classification model, to list a few examples.

One or more automated state-based actions are performed according to the disease state determined for the plant. In one example, these actions comprise updating an area-of-interest map to indicate the disease state of one or more plants positioned in an area-of-interest and presenting the area-of-interest map via displays of one or more user devices. Here, the reflectance signal data for the plant may have been collected via a sensing platform comprising one or more data collection devices configured to acquire reflectance signal data of plants within the area of interest. These data collection devices are configured to record position and orientation data associated with the collected reflectance signal data. In this case, the area-of-interest map is generated and/or updated based at least in part on the recorded position and orientation data associated with the collected reflectance signal data.

In general, according to another aspect, the invention features a system for detecting diseases in plants. The system comprises a disease classification system that determines a disease state of the plant. It does this by receiving reflectance signal data for a plant, identifying a plurality of signal components of the reflectance signal data, selecting, from the plurality of signal components, signal components having a variance satisfying a variance threshold. The variance is between particular signal components of reflectance signal data for a non-diseased plant of the plant species and corresponding signal components of reflectance signal data for a diseased plant of the plant species. The disease classification system then generates reduced-spectrum frequency data based at least in part on the reflectance signal data and the selected components, generates a reflectance signature for the plant based at least in part on the reduced-spectrum frequency data, and determines a disease state of the plant based at least in part on the reflectance signature.

In general, according to another aspect, the invention features a computer program product comprising at least one non-transitory computer-readable storage medium having computer-readable program code portions stored therein. The computer-readable program code portions comprise an executable portion configured to receive reflectance signal data for a plant, an executable portion configured to identify a plurality of signal components of the reflectance signal data, and an executable portion configured to select signal components from the plurality of signal components, the selected signal components having a variance satisfying a variance threshold. Here, the variance is between particular signal components of reflectance signal data for a non-diseased plant of the plant species and corresponding signal components of reflectance signal data for a diseased plant of the plant species. The computer-readable program code portions further comprise an executable portion configured to generate reduced-spectrum frequency data based at least in part on the reflectance signal data and the selected signal components, an executable portion configured to generate a reflectance signature for the plant based at least in part on the reduced-spectrum frequency data, and an executable portion configured to determine a disease state of the plant based at least in part on the reflectance signature.

### BRIEF DESCRIPTION OF THE SEVERAL VIEWS OF THE DRAWING(S)

Having thus described embodiments of the present disclosure in general terms, reference will now be made to the accompanying drawings, which are not necessarily drawn to scale.

FIG. 1 provides an exemplary overview of an example system architecture that may be used to practice various embodiments of the present disclosure;

FIG. 2 is a schematic of an example system for detecting and classifying a disease state of a plant based at least in part on generating a reflectance signature for the plant, in accordance with various embodiments of the present disclosure;

FIG. 3 provides a process flow of example operations for detecting and classifying a disease state of a plant, in accordance with various embodiments of the present disclosure;

FIG. 4A and FIG. 4B illustrate exemplary image capturing devices of a sensing platform that may be used to practice various embodiments of the present disclosure;

FIG. 5A illustrates an exemplary image capturing device of the sensing platform;

FIG. 5B illustrates exemplary leaf samples for which reflectance signal data is collected by the image capturing device illustrated in FIG. 5A;

FIG. 6 illustrates exemplary citrus leaf samples at various disease stages, for which reflectance signal data is collected in accordance with various embodiments of the present disclosure;

FIG. 7 illustrates exemplary avocado leaf samples at various disease stages, for which reflectance signal data is collected in accordance with various embodiments of the present disclosure;

FIG. 8 illustrates a two-dimensional plot of the first four eigenfunctions of an exemplary function, in accordance with various embodiments of the present disclosure;

FIG. 9 illustrates an expanded series function for a Mercer's Theorem expansion of an equation, in accordance with various embodiments of the present disclosure;

FIG. 10A and FIG. 10B illustrate exemplary frequency data generated in determining a reflectance signature for a plant;

FIG. 11A and FIG. 11B illustrate exemplary reduced-spectrum frequency data generated in determining a reflectance signature for the plant;

FIG. 12A and FIG. 12B illustrate example reduced-spectrum frequency data generated in determining a reflectance signature for the plant;

FIG. 13A and FIG. 13B illustrate example reflectance signatures for one or more plants that may be used in detecting and classifying disease states of the plants; and

FIG. 14 illustrates exemplary reflectance signatures overlaid a signature generated based at least in part on non-reduced frequency data reflecting a complete spectrum within a frequency range;

FIGS. 15A and 15B show exemplary signal envelopes for reflectance signatures, the envelopes representing averaged signatures that can be used as representative biomarkers for plants;

FIG. 16 illustrates an exemplary classification operation for detecting and classifying disease states of a plant, in accordance with various embodiments of the present disclosure;

FIG. 17 illustrates an exemplary classification operation for detecting and classifying disease states of a plant, in accordance with various embodiments of the present disclosure; and

FIG. 18 illustrates an exemplary screen of a graphical user interface for presenting disease state information in accordance with various embodiments of the present disclosure.

### DETAILED DESCRIPTION

Various embodiments of the present disclosure now will be described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

### I. General Overview and Technical Advantages

Various embodiments of the present disclosure are directed to detecting and classifying a disease state of a plant based at least in part on generating a reflectance signature for the plant from reflectance signal data. The reflectance signal data may be generated using hyperspectral imaging sensors, in various instances. The reflectance signature is specifically generated based at least in part on determining and using a reduced spectrum for the reflectance signal data and extracting data from frequency components with the highest energy and/or variance. In various embodiments, Karhunen-Loeve Expansion (KLE) techniques are used to identify the frequency components that represent the distribution of the reflectance signal data, and spectral decomposition techniques are performed to determine eigenvalues associated with the frequency components. Such eigenvalues are used to identify and select the frequency components that comprise the highest variation in the reflectance signal data. Thus, these identified and selected frequency components (e.g., high-variance frequency components) may be understood, by those of skill in the field to which the present disclosure pertains, as the weighted variables that carry most of the information on the reflectance signal data, or the reflectance spectrum, of the plant.

In various embodiments, frequency reconstruction techniques are efficiently performed (e.g., due to the identification and reduction to significant high-variance data) with the high-variance frequency components to generate reduced-spectrum frequency data. For example, information carried by the high-variance frequency components are converted to a wave function. In some embodiments, the frequency reconstruction techniques include Fast Fourier Transform techniques. The reduced-spectrum frequency data may then be used to generate a reflectance signature for the plant, for further use in non-or less-invasive plant identification, classification, and disease biomarking.

Thus, various embodiments provide various technical advantages in disease detection and classification for plants. By examining reflectance signal data, inherent biomarkers in plants are examined, thereby providing a deeper and more accurate analysis compared to visual analysis. Such examination using reflectance signal data enables earlier detection and classification of disease, as visual manifestation of symptoms for various diseases may only occur in later disease stages. Further, various embodiments involve reduction of reflectance signal data to high-energy and high-variance frequency components that carry the majority of information of the reflectance signal data. This reduction enables improved efficiency in computational and processing resources, due to there being less data and fewer dimensions thereof to analyze.

### II. Exemplary System Architectures

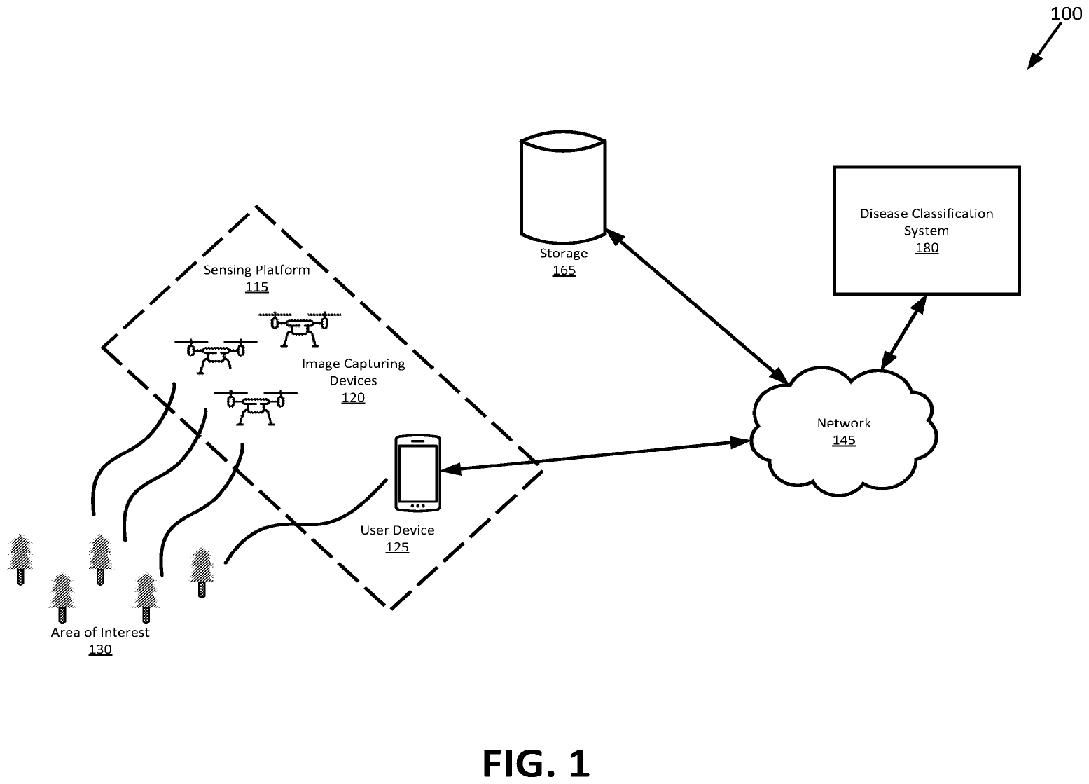

FIG. 1 provides an illustration of a system architecture 100 that may be used in accordance with various embodiments of the disclosure. Here, a sensing platform 115 is used for data and/or image acquisition of an area of interest 130 that includes one or more data collection devices 120 configured to acquire reflectance signal data of the area of interest 130. The area of interest 130 may include one or more plants of different disease states. In various embodiments, the data collection devices 120 are and/or comprise quadcopter UAVs, such as a DJI Matrice 600 Pro Hexacopter, for example. The data collection devices 120 comprise data collection means for collecting reflectance signal data from plants in the area of interest 130. In various embodiments, such data collection means include hyperspectral cameras (e.g., a Resonon Pika L 2.4 hyperspectral camera, Spectronon Pro, Resonon, Bozeman, MT), visible-near infrared (V-NIR) objective lenses, and/or the like. In various embodiments, the visible-near infrared objective lenses have a focal length of 23 mm, field of view of 13.1 degrees, and instantaneous field of view of 0.52 mrad. Each data collection device 120 is also configured with a global positioning system and an inertial measurement unit to record position and orientation data. In some embodiments, position and orientation data is associated with the reflectance signal data.

In various instances, the sensing platform 115 is configured to collect reflectance signal data for a particular plant, and specifically for individual leaves thereof. For example, a data collection device 120 is a hyperspectral camera, a spectroradiometric sensor, a device comprising a hyperspectral camera and/or a spectroradiometric sensor, and/or the like. When collecting reflectance signal data for individual leaves, halogen light sources may be used to create optimal conditions, and spectral signatures may be calibrated with a barium sulphate standard reflectance panel.

In addition, the sensing platform 115 may include a user device 125 for controlling the data collection devices 120, receiving and/or storing data from the data collection devices 120, and/or the like. That is, the reflectance signal data may be collected on the user device 125. In various embodiments, the user device 125 stores the reflectance signal data and/or provides the reflectance signal data to a storage system 165 (e.g., a database) for storage.

As illustrated in FIG. 1, the system architecture 100 further comprises a disease classification system 180, and the user device 125 may provide the reflectance signal data to the disease classification system 180, in some embodiments. In various embodiments, the disease classification system 180 is configured to detect and classify a disease state of a plant using the reflectance signal data for the plant. In particular, the disease classification system 180 is configured to generate a reflectance signature for the plant and use the reflectance signature to classify a disease state of the plant.

In some embodiments, the disease classification system 180 may communicate with the user device 125, the storage system 165, and/or other various entities using one or more communication networks 145. Examples of communication networks 145 include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like). In various embodiments, the disease classification system 180 comprises an application programming interface (API), receives reflectance signal data for a plant via an API call, and provides a classified disease state of the plant via an API response.

The storage system 165 may be configured to store data for detecting and classifying disease states for plants, including reflectance signatures generated by the disease classification system 180. The storage system 165 may include one or more storage units, such as multiple distributed storage units that are connected through a computer network. Each storage unit in the storage system 165 may store at least one of one or more data assets and/or one or more data about the computed properties of one or more data assets. Moreover, each storage unit in the storage system 165 may include one or more non-volatile storage or memory media including, but not limited to, hard disks, solid state disks or drives, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

Moreover, in various embodiments, the disease classification system 180 is configured to perform one or more automated state-based actions according to the classified disease states of the plants as determined by the disease classification system 180. In one example, the disease classification system 180 updates an area-of-interest map to indicate the disease state of one or more plants positioned within an area of interest (e.g., based at least in part on position and orientation data associated with the reflectance signal data) and presents this area-of-interest map via displays of one or more user devices 125, including possibly the user devices 125 used to control the sensing platform 115 or any other user devices 125 connected to the disease classification system 180 via the network 145. In another example, the disease classification system 180 pushes alerts to one or more user devices 125 in response to the disease classification system 180 detecting certain disease states. These alerts can include information identifying detected disease state and/or location information indicating where the plants with the disease states were detected based at least in part on position and orientation data associated with the reflectance signal data.

### III. Exemplary Computing Entities

In general, the terms device, system, computing entity, entity, and/or similar words used herein interchangeably can refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

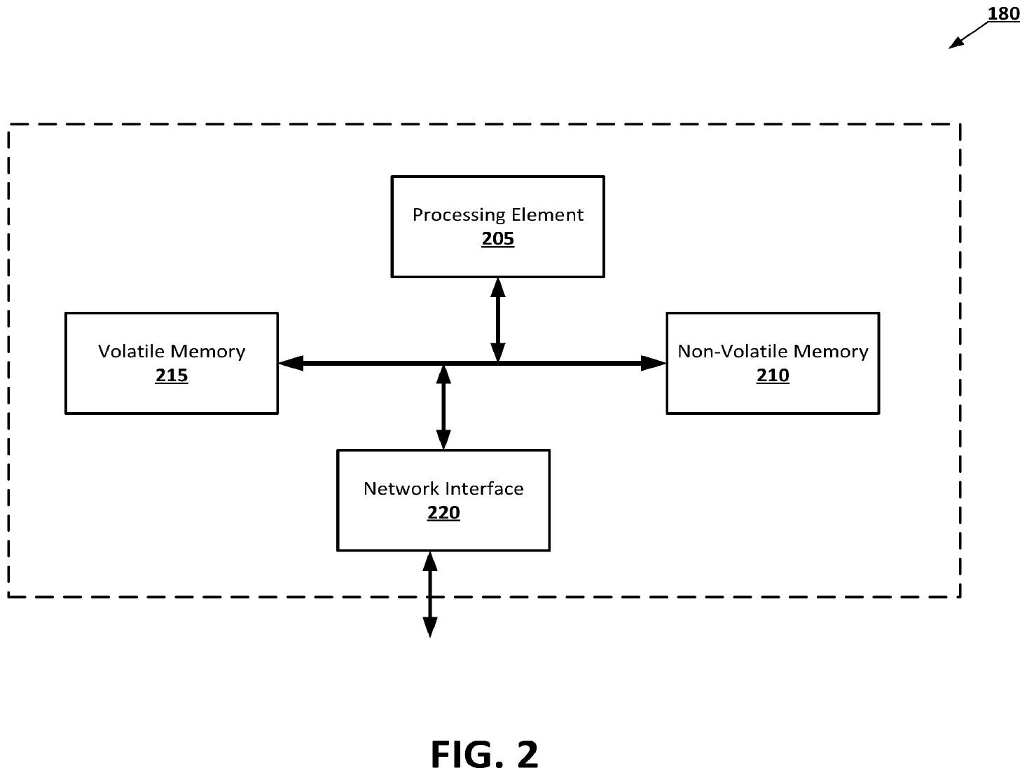

FIG. 2 provides an illustrative schematic representative of a disease classification system 180 that can be used in conjunction with embodiments of the present disclosure. For instance, the disease classification system 180 may be configured to and/or comprise means for obtaining reflectance signal data for a plant, determining or generating a reflectance signature for the plant, generating, updating, and/or maintaining a signature database (e.g., storage system 165) for reflectance signatures, classifying disease states of plants using reflectance signatures, and/or the like. As shown in FIG. 2, in one embodiment, the disease classification system 180 may include, or be in communication with, one or more processing elements 205 (also referred to as processors, processing circuitry, and/or similar terms used herein interchangeably) that communicate with other elements within the disease classification system 180 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In one embodiment, the disease classification system 180 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media 210 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the disease classification system 180 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media 215 may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the disease classification system 180 with the assistance of the processing element 205 and operating system.

As indicated, in one embodiment, the disease classification system 180 may also include one or more network interfaces 220 for communicating with various computing entities (e.g., one or more user devices 125) such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the disease classification system 180 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the disease classification system 180 may include, or be in communication with, one or more input elements, such as a keyboard input, a mouse input, a touch screen/display input, motion input, movement input, audio input, pointing device input, joystick input, keypad input, and/or the like. The disease classification system 180 may also include, or be in communication with, one or more output elements (not shown), such as audio output, video output, screen/display output, motion output, movement output, and/or the like.

As will be appreciated, one or more of the components of the disease classification system 180 may be located remotely from other components, such as in a distributed system. Furthermore, one or more of the components may be aggregated and additional components performing functions described herein may be included in the disease classification system 180. Thus, the disease classification system 180 can be adapted to accommodate a variety of needs and circumstances.

### IV. Exemplary System Operations

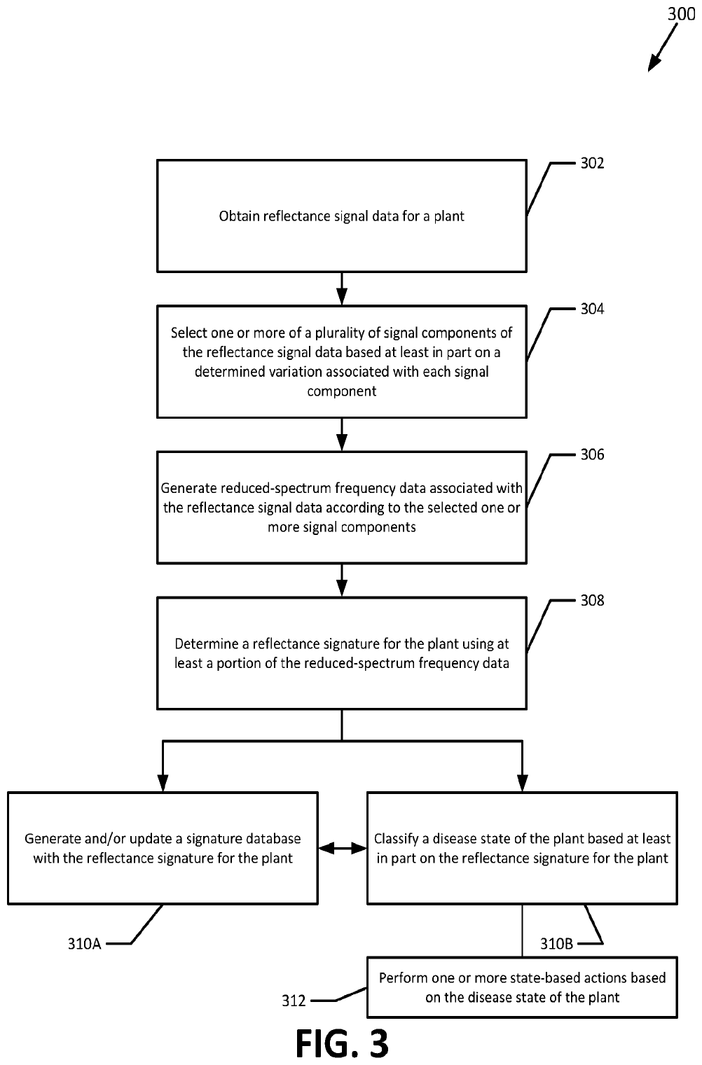

FIG. 3 illustrates a process 300 for detecting and classifying a disease state of a plant based at least in part on generating a reflectance signature for the plant. In various embodiments, operations of process 300 may be performed by the disease classification system 180, and the disease classification system 180 comprises means, such as processing element 205, memories 210, 215, network interface 220, and/or the like, for performing the operations of process 300.

As illustrated in FIG. 3, process 300 comprises operation 302, and in one embodiment, process 300 begins with operation 302. Operation 302 comprises obtaining reflectance signal data for a plant. Reflectance signal data for a plant is generated using sensing platform 115, which comprises one or more image capturing devices 120, as previously described with respect to FIG. 1. An image capturing device 120 may be a stand-alone hyperspectral camera or spectroradiometric sensor or may be a hyperspectral camera or spectroradiometric sensor attached to a UAV for aerial imaging. In various embodiments, reflectance signal data for the plant is generated and directly provided to the disease classification system 180, such that the disease classification system 180 obtains the reflectance signal data for the plant in real-time. In other embodiments, the reflectance signal data for the plant may be stored (e.g., in storage system 165 as a dataset), and the disease classification system 180 is configured to access and/or retrieve the reflectance signal data. In some embodiments, the disease classification system 180 is configured to operate UA Vs of the sensing platform 115 in order to obtain reflectance signal data from a plant in real-time.

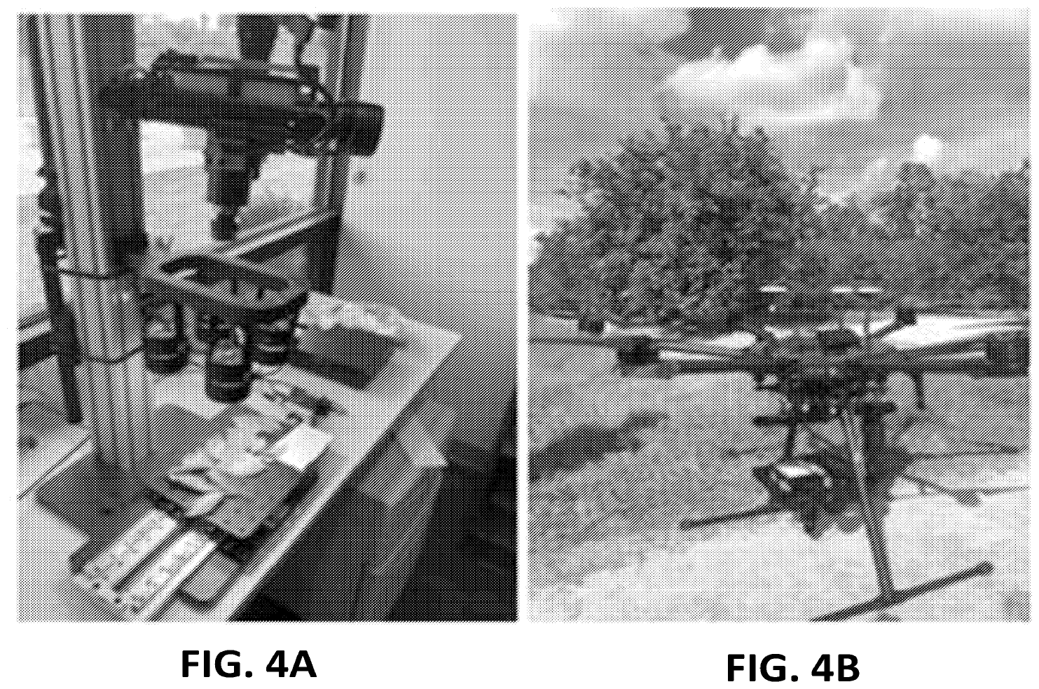

In general, FIGS. 4A and 4B show different examples of the image capturing devices 120 of the sensing platform 115, including exemplary image capturing devices 120 of a hyperspectral sensing system. In the illustrated examples, FIG. 4A shows a hyperspectral camera, namely a Resonon Pika L2 in a laboratory setting, and FIG. 4B shows a similar hyperspectral camera attached to a UAV.

In one example, spectral data can be collected for a citrus dataset by a spectroradiometer (Resonon Pika L2, Integrys, Mississauga, ON) with 4° fields of view in laboratory conditions. The wavelengths ranges between 400-1,000 nm, with an average spectral resolution of 1.3 nm. Four 3-inch diameter LED light sources are installed on two sides to improve ideal conditions for performing scans and reducing errors, as shown in FIG. 4A. The Resonon Pika L2 device is situated so that the lens is 0.5 m above the sample, at a direct line of sight of the specimens. Spectral signatures are calibrated with a barium sulfate standard reflectance panel (Spectral Reflectance Target, CSTM-SRT-99-100, Spectra Vista Cooperation, NY) before and immediately after every eight sample measurements. Five scans per leaf (five locations on each leaf) for each healthy (H) and CC infected plant are collected.

In another example concerning the citrus plant, spectral data is collected using a UAV (DJI Matrice 600, Pro Hexacopter, China) equipped with a hyperspectral camera, as shown in FIG. 4B. The UAV-based imaging system includes: (i) a Resonon Pika L 2.4 hyperspectral camera (Spectronon Pro, Resonon, Bozeman, MT); (ii) visible-near infrared (V-NIR) objective lenses for the Pika L camera with a focal length of 23 mm, field of view (FOV) of 13.1 degrees, and instantaneous field of view (IFOV) of 0.52 mrad; and (iii) a global positioning system (GPS) and the inertial measurement unit (IMU). The IMU flight control system for multi-rotor aircraft is used to record sensor position and orientation. In this case, data is collected at 30 m above the ground with a speed of 1.5 mph. The positions of the infected trees are known (e.g., leaves were previously collected and identified by polymerase chain reaction (PCR) as described in Abdulridha et al. (2019)). The maps and images are analyzed by the Spectronon software after spectral data is acquired. Calibration corrections are performed using the Resonon hyperspectral data analysis software (Spectronon Pro, Resonon, Bozeman, MT). Georectification and radiometric correction plugins from the Spectronon Pro software are used to correct the GPS/IMU and the radiometric data, respectively. Regions of interest are selected manually and randomly for each tree, and twenty spectral scans are performed to ensure that the entire canopy is covered spectrally. The regions of interest are then exported as a text file. Pixel-based reflectance data are collected for each class of plant (healthy and CC affected at the asymptomatic, early, and late disease development stages).

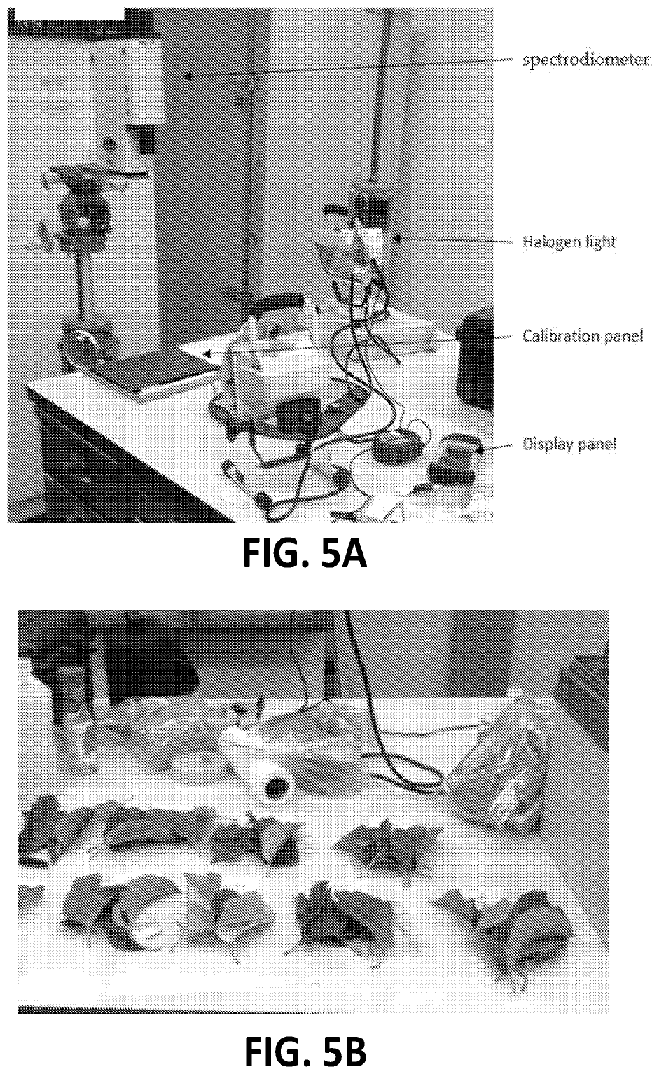

In another example concerning avocado plants, five scans per leaf are taken for each healthy, Lw infected, and N, Fe deficient sample. These samples are collected between 350 and 2,500 nm utilizing a spectroradiometric sensor (SVC HR-1024, Spectra Vista Cooperation, NY) with 1.3 nm average spectral resolution and 4° fields of view in laboratory conditions. According to this example, FIG. 5A shows an example of the spectroradiometric sensor, and FIG. 5B shows exemplary avocado leaf samples in early and late stages. Only the spectral range of 400-970 nm is used for data analysis. Two halogen lights are used to create optimal conditions for performing the scans without a natural light source. The SVC device is situated so that the lens is 0.8 m above the leaf samples. Spectral signatures are calibrated with a barium sulfate standard reflectance panel (Spectral Reflectance Target, CSTM-SRT-99-100, Spectra Vista Cooperation, NY) before and immediately after every eight sample measurements. Black fabric is utilized as background.

The disease classification system 180 obtains the reflectance signal data for non-diseased (e.g., healthy) plants and diseased plants, which include plants infested with particular diseases and/or plants having abiotic stressors that mimic disease symptom properties in the reflectance spectrum.

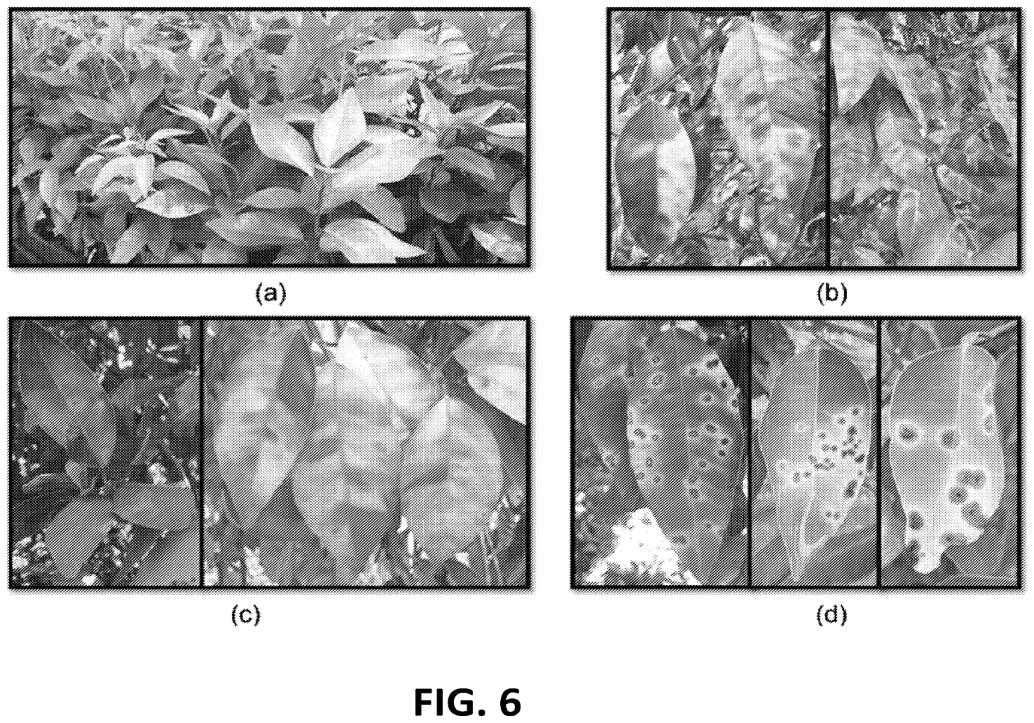

In one example, the reflectance signal data is obtained for healthy citrus plants (e.g., Sugar Belle mandarin) and those infected with citrus canker (CC) diseased states at several stages of the disease infestation (e.g., asymptomatic, early, late). Examples of this tree and stages of the citrus canker infected leaves are shown in FIG. 6. More particularly, in FIG. 6, item (a) shows healthy Sugar Belle mandarin leaves. On the other hand, item (b) shows asymptomatic Sugar Belle mandarin leaves (e.g., no canker lesion). It is important to note that these leaves have HLB disease-associated blotch mottling symptoms that are ubiquitous in citrus in Florida and different than those caused by CC. Item (c) shows CC-infected leaves at an early developmental stage of the disease, and item (d) shows CC-infected leaves at a late developmental stage of the disease.

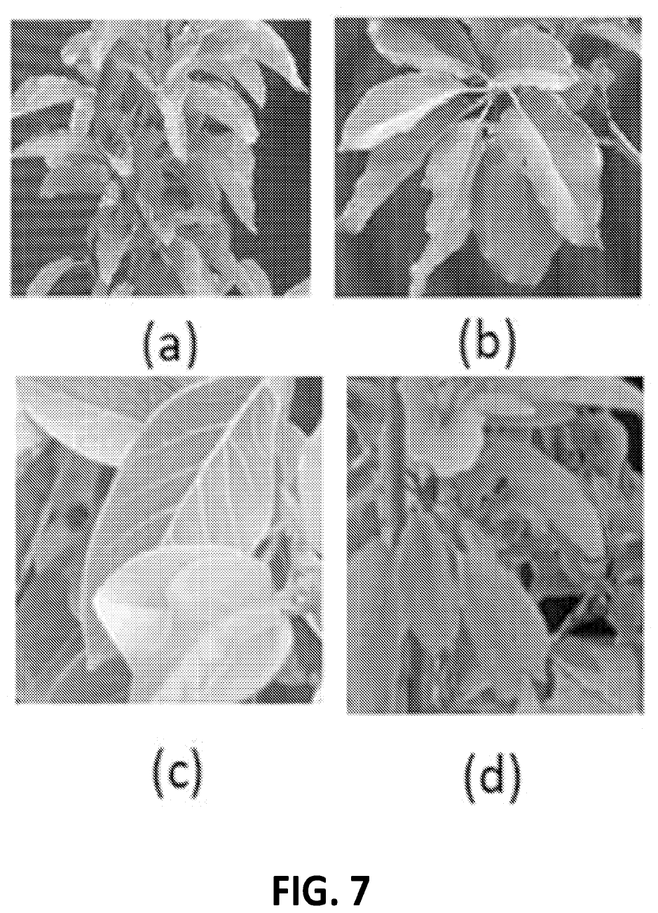

In another example, the reflectance signal data is obtained for avocado trees, including non-diseased trees and those infected with Laurel wilt (Lw) and those having iron (Fe) and nitrogen (N) deficiencies. Because iron (Fe) and nitrogen (N) deficiencies in the avocado present similar symptoms as the Lw infected avocado, additional data from the Fe and N deficient plants were collected, as shown in FIG. 7. More particularly, FIG. 7 shows examples of avocado leaves used in the study, including healthy leaves at item (a), leaves with N deficiency at item (b), leaves with Fe deficiency at item (c), and Lw-infected leaves at item (d).

In one exemplary scenario, the disease classification system 180 obtains the reflectance signal data for plants with known disease states in order to perform a training process, in which case the reflectance signal data is labelled or tagged to indicate the known disease state of the plant.

In another exemplary scenario, the disease classification system 180 obtains the reflectance signal data for plants with unknown disease states in order to classify the disease states of the plants.

In various embodiments, obtaining reflectance signal data for the plant comprises processing the reflectance signal data (e.g., in preparation for generation of a reflectance signature and classifying a disease state for the plant). For example, Standard Normal Transformation techniques are performed to provide preservation of data integrity and to restructure the data into a reasonable population domain.

In one example, leaf reflectance data are kept in a matrix form with the columns representing wavelengths (j=386.8(1),900.2(N)), where the first wavelength starts at 386.8 nm, and the last wavelength is 900.2 nm (N). Each vector wavelength increments by ˜1.3 nm. The row data represents leaf samples (i=1, M). Each column vector of the matrix, X, is the reflectance of the particular leaf sample at the column wavelength (j). In this case, an exemplary leaf sample collected for leaf five at 900.2 nm would be given mathematically as X (5, N).

An exemplary probability density function of the Standard Normal transformation is provided in equation (1):

$$f ( X \vert μ , σ ) = \frac{1}{\sqrt{2 π {σ}^{2}}} {e}^{- ζ} \\ Where \qquad ( 1 )$$

$$ζ = \frac{{( X - μ )}^{2}}{2 σ} \qquad ( 2 )$$

represents the normal distribution parameter; u represents the mean of the population (measured reflectance data for each leaf category analyzed) and o represents the standard deviation. Using Standard Normal Transformation techniques, the reflectance signal data is normalized and is then used for the generation of a reflectance signature.

Returning to FIG. 3, process 300 further comprises operation 304, which comprises selecting one or more of a plurality of signal components of the reflectance signal data based at least in part on a determined variance associated with each signal component. In one example, the variance is between particular signal components of reflectance signal data for non-diseased plants of the plant species and corresponding signal components of reflectance signal data for diseased plants of the plant species. Here, the selected signal components have a variance satisfying a variance threshold, which generally is a calculated or predetermined threshold suitable for identifying the most relevant information within the reflectance signal data for classifying disease states for each plant species.

In various embodiments, techniques in accordance with Mercer's theorem are performed to generate signal components, frequency components, orthonormal basis functions, orthonormal modes, eigenvectors, eigenfunctions, and/or similar terms used interchangeably herein, from the reflectance signal data. In general, Mercer's Theorem states that given a symmetric, positive kernel function, (F(x, y)), that is continuous on [a,b] x [a,b], then there exists a corresponding orthogonal set of eigenfunctions, ψₖ(x), and eigenvalues, λₖ. In one example, Mercer's Theorem is applied to decompose the reflectace signal data into orthonormal basis functions, as given in equation (3):

$$F ( x , y ) = {\sum }_{k = 1}^{∞} {λ}_{k} {ψ}_{k} ( x ) {ψ}_{k} ( y ) \\ Where {λ}_{k} = eigenvalues of F ( x , y ) {ψ}_{k} ( x ) = eigenfunctions of F ( x , y ) {ψ}_{k} ( y ) = symmetric eigenvectors of F ( x , y ) \qquad ( 3 )$$

By the orthogonality property for orthonormal basis functions, (3) can be rewritten as:

$${λ}_{k} {ψ}_{k} ( x ) = {\int }_{a}^{b} F ( x , y ) {ψ}_{k} ( y ) d y \qquad ( 4 )$$

Here, because the data is normalized, the mean is zero and is therefore not included in the expansion. Moreover, the arguments taken for the kernel function are the actual plant reflectance values and the wavelengths.

In various embodiments of the present disclosure, a transformation into frequency components from wavelength data is performed to provide signature data in the frequency domain.

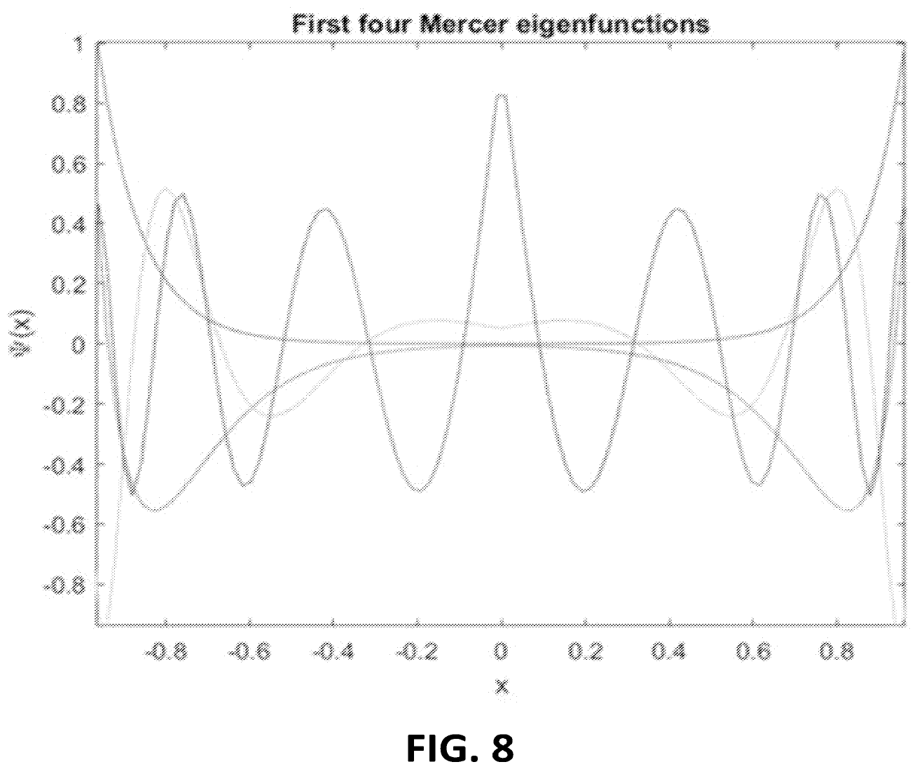

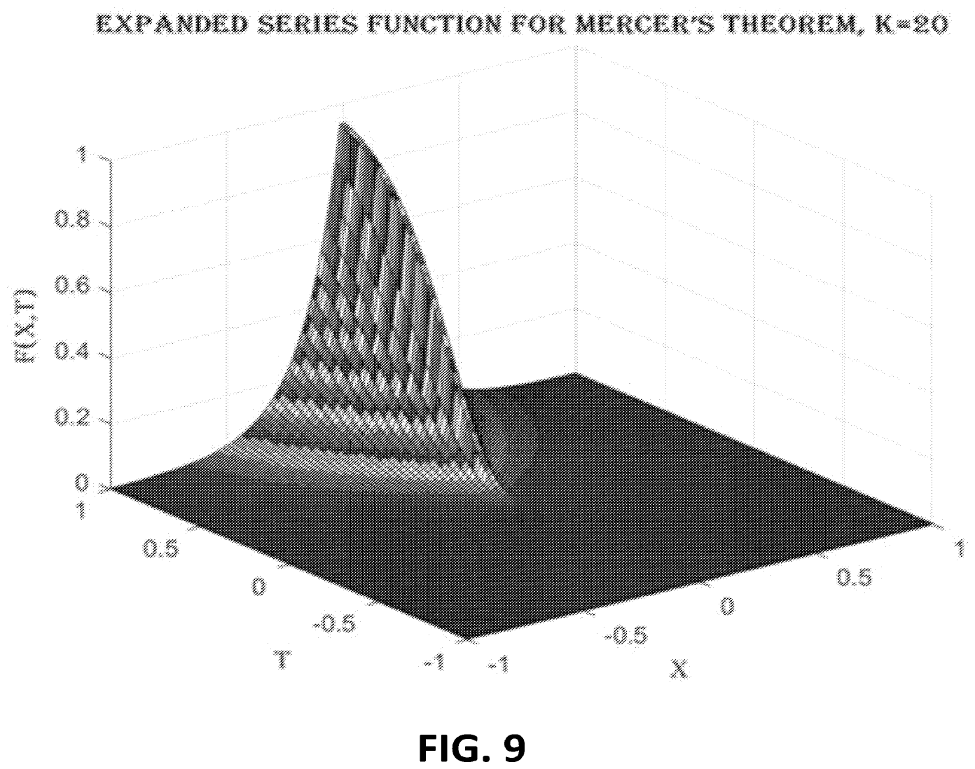

In an exemplary function F(x,y)=e^{-2π|(x}^{^{y}}^{)|} for x=[−1, 1], y=[−1, 1], the first four eigenfunctions are given by a two dimensional plot, provided in FIG. 8, which shows the first four Eigenfunctions for a Mercer's Theorem expansion of Equation 3. FIG. 9 shows the expanded series function for a Mercer's Theorem expansion of Equation 3.

In this case, orthogonality/orthonormality can be proven by multiplying the eigenfunction by its transpose. For the first six eigenvectors:

$${{ψ}_{k} ( x )}^{T} {ψ}_{k} ( y ) = \begin{matrix}1 & 0 & 0 & 0 & 0 & 0 \\ 0 & 1 & 0 & 0 & 0 & 0 \\ 0 & 0 & 1 & 0 & 0 & 0 \\ 0 & 0 & 0 & 1 & 0 & 0 \\ 0 & 0 & 0 & 0 & 1 & 0 \\ 0 & 0 & 0 & 0 & 0 & 1\end{matrix}$$

Here, truncating the series leads to a loss of accuracy but still preserves over 98.1% of the relevant data within the first four terms:

$$F ( x = 0 , y = 0 ) = {\sum }_{k = 1}^{4} {λ}_{k} {ψ}_{k} ( x ) {ψ}_{k} ( y ) = 0.981 \qquad ( 5 )$$

Moreover, in various embodiments, Mercer's Theorem can be expanded to use the covariance function as the kernel function. Specifically, the Karhunen-Loeve Expansion (KLE) is used to describe stochastic processes that are related by the mean and covariance. Equation (6) accordingly provides the decomposition of reflectance signal data into eigenvectors using KLE with Mercer's Theorem:

$$X ( x , f ) = μ ( x ) + {\sum }_{j = 1}^{N} K ( x , {f}_{j} ) Z ( x , {f}_{j} ) \qquad ( 6 )$$

In equation (6), x represents spatial point intensity, fj represents frequency of decomposition, X(x,f) represents a multivariate, stochastic process equation, μ(x) represents a mean value at x, K(x,f) represents covariance represented by λₖψₖ(x), and Z(x,f) represents uncorrelated eigenfunctions of unit variance.

The KLE is an optimized transformation along all orthogonal component vectors. The KLE process can be used to determine a formal decorrelation of signal energy of the reflectance signal data into a redistribution that weighs more heavily the components of highest energy contribution (or disturbances) for a particular system. That is, the KLE techniques realize the lowest order data that adequately describes the main functional contributors to the reflectance signal data and/or the equilibrium of a system and its cellular dynamics reflected in the plant reflectance spectrum. When disease or deficiencies infiltrate a cell, this disturbance is shown by its variance in the reflectance spectrum at specific frequencies.

The multivariate approach (e.g., detailed in Equation 6) uses several spectral bands, with the X-variate matrix (e.g., X(x, λ)) being based at least in part on twenty wavebands, in some embodiments. In various embodiments, a cross-covariance matrix is derived through variance (e.g., σᵢ²) and covariance (e.g., σᵢⱼ) of the X-variate matrix (e.g., X(x, λ)). The eigenvectors are used to distinguish the modal components associated with the variance between infected and healthy plants. In various embodiments, the X-variate matrix is obtained for reflectance signal data at varying wavelengths, in accordance with Equation 7:

$$X = ( \begin{matrix}{x}_{11} & … & {x}_{1 n} \\ ⋮ & ⋱ & ⋮ \\ {x}_{m 1} & … & {x}_{mn}\end{matrix} ) , or X = [ {x}_{1} , {x}_{2} , … {x}_{n} ] \qquad ( 7 )$$

In some embodiments, the data for the reflection coefficients are given by the matrix elements and represent data in the wavebands from 348-2505 nm. In various embodiments, the cross-covariance matrix is then obtained from cov(xᵢ,xⱼ)=E[(xᵢ−x̅ᵢ)(xⱼ−x̅ⱼ)^{T}]=σₓ_{ᵢ}σₓ_{ⱼ} and from Equation 8:

$$K = [ \begin{matrix}{σ}_{{x}_{1}}^{2} & … & {σ}_{{x}_{1}} {σ}_{{x}_{n}} \\ ⋮ & ⋱ & ⋮ \\ {σ}_{{x}_{n}} {σ}_{{x}_{1}} & … & {σ}_{{x}_{n}}^{2}\end{matrix} ] \qquad ( 8 )$$

Here, σ represents the covariance of the x independent variates.

Equation 9 provides the Spectral Decomposition Theorem, which may be applied to further process the cross-covariance matrix:

$$K {ε}_{i} = {λ}_{i} {ε}_{i} \qquad ( 9 )$$

In this equation, the λᵢ represent the eigenvalues that correspond to the eigenvectors of the cross-covariance matrix, and εᵢ represent the eigenvectors of the estimated linear coefficients.

Accordingly, an estimate of the covariance matrix can be approximated and applied as described by Equation 10:

$$Y = {ε}_{i 1} {x}_{1} + {ε}_{i 2} {x}_{2} + … {ε}_{i n} {x}_{n} \qquad ( 10 )$$

Here, the variance for the Yᵢ component of the linear transformation model is equal to the variance of the corresponding eigenvector equation, which is equal to its eigenvalue, as shown in Equation (11):

$$var ( {Y}_{i} ) = var ( {e}_{i 1} {x}_{1} + {e}_{i 2} {x}_{2} + … {e}_{i n} {x}_{n} ) = {λ}_{i} \qquad ( 11 )$$

An approximation matrix, \hat{Z}, can be formulated in accordance with the explained percentage of the variance given by Equation (11), as shown in Equation (12):

$$L = \frac{{\sum }_{i = 1}^{ℓ} {λ}_{i}}{{\sum }_{j = 1}^{n} {λ}_{j}} \qquad ( 12 )$$

In Equation (12), [symbol] represents a number of components, or eigenvectors, that is less than the total count of components. The denominator of L is also known as the trace and equals the sum of the eigenvalues. In various embodiments, maximation of L while minimizing l provides a reduced or lower-order representation of the reflectance signal data while retaining a maximal amount of variance. That is, l represents the optimal number of signal components, or eigenvalues, necessary to accurately approximate the reflectance signal data. Here, the goal is to maximize L with the least number of modal components of the numerator.

The approximation matrix \hat{Z} may then be determined using Equation (13):

$$\hat{Z} = {\sum }_{i = 1}^{ℓ} {ε}_{i} Y = {\sum }_{i = 1}^{ℓ} {ε}_{i} {ε}_{i}^{T} X = {I}_{l} X \qquad ( 13 )$$

In Equation 12, Iₗ represents the l-th order identity matrix. Altogether then, an estimate of the X-variate matrix with a reduced set of coefficients through applying Mercer's Theorem can be achieved through Equation (14):

$$X ( x , f ) = μ ( x ) + {\sum }_{i = 1}^{ℓ} K ( x , {f}_{i} ) \hat{Z} ( x , {f}_{i} ) \qquad ( 14 )$$

It will be appreciated by those of skill in the field to which the present disclosure pertains that the \hat{Z} matrix is a reconstructed KLE approximation of the original multivariate reflectance signal data and contains only the pertinent signal components of the data minus the extraneous noise and other factors that account for less than (100−L)% of the data. The eigenvectors associated with the \hat{Z} matrix are fundamentally important and used in generating a reflectance signature for the plant.

In this way, with at least the above, a plurality of signal components of the reflectance signal data are identified, and signal components from the plurality of signal components are selected, with the selected signal components having a variance satisfying a variance threshold, as indicated in step 304 of FIG. 3. More particularly, a plurality of signal components, or eigenvectors, of the reflectance signal data may be determined, and a variance associated with each signal components may be determined. Further, one or more of the plurality of signal components (e.g., l number of signal components) are selected according to the percentage of the variance associated with each signal component out of the total variance. In some embodiments, l is four, and four signal components are selected. In other embodiments, l is three, and three signal components are selected. In various embodiments, l is determined based at least in part on Equation 11 and a configurable threshold for L. For example, l number of signal components are selected to carry approximately 96% of the variance of the reflectance signal data (e.g., L=0.96).

In one example, for citrus plants, a KLE process is performed to determine that the first four modal components were found to carry ˜96% of the effective eigenvalues. In a similar example, a KLE process is performed to determine that the first fourteen modal components accounted for about 67% of the cumulative contribution of variance.

In various embodiments, the selected signal components are based at least in part on determined variance in the reflectance signal data for plants of a particular species and are determined specifically for particular plant species.

Returning to FIG. 3, at operation 306, reduced-spectrum frequency data is generated. The reduced-spectrum frequency data is generated from the reflectance signal data using the selected one or more signal components. In various embodiments, the reflectance signal data is generated and/or obtained in the frequency domain; that is, the reflectance signal data is given per wavelength. Accordingly, the reflectance signal data is translated to the frequency domain, and frequency decomposition is performed, in various embodiments. Thus, in various embodiments, generating reduced-spectrum frequency data comprises generating frequency data from the reflectance signal data and reducing the frequency data based at least in part on the selected one or more signal components (e.g., l number of eigenvectors).

In various embodiments, frequency data is first generated (e.g., full-spectrum frequency data) according to inverse Fourier Transform techniques. For a specific period of the reflectance signal data, Fourier coefficients are obtained via the inverse Fourier Transform techniques. In some embodiments, an inverse Fourier Transform of the data is carried out by sampling N=4096 data points in the region of normalized frequency (0≤ω≤2π). A truncated series expansion using Fourier Transform techniques is then provided by Equations 15 and 16:

$$x ( y ) = \frac{1}{N} {\sum }_{n = 0}^{N - 1} {a}_{n} {e}^{- 2 π j {f}_{n} y} \qquad ( 15 )$$

$$x ( {y}_{m} ) = \frac{1}{N} {\sum }_{n = 0}^{N - 1} {a}_{n} {e}^{- 2 π j {f}_{n} {y}_{m}} for m = 0 , \frac{1}{{f}_{s}} , \frac{2}{{f}_{s}} , … \frac{( N - 1 )}{{f}_{s}} \qquad ( 16 )$$

In the above equations, f₀ represents the lowest frequency, fₛ represents the sampling rate, and fₙ=nf₀, n=0, 1, . . . , N−1. Additionally, yₘ represents discrete points, and aₙ represent Fourier coefficients.

Equation 17 then provides a definition of xₙ for the sampled frequency data:

$$( {x}_{n} ) = {\sum }_{n = 0}^{N - 1} {A}_{n m} {a}_{n} \qquad ( 17 )$$

Further, Equation 18 provides an approximation \hat{x} of the frequency vector of xn values:

$$\hat{x} = A \hat{a} \qquad ( 18 )$$

In Equation 18, A represents sampled frequency data amplitudes, and â represents approximation Fourier coefficients, which can be solved for through matrix inversion. Solving for the coefficient vector results in Equation 19:

$$\hat{a} = {A}^{- 1} \hat{x} \qquad ( 19 )$$

In various embodiments, this method can be applied for various expansion sets, and/or frequency data may be generated similar to the above using Fast Fourier Transform (FFT) techniques. In various embodiments, a wavelet packet transform may be used to apply a similar nonlinear scalar wave function to form a complete orthonormal basis system of functions. Other orthogonal basis sets can also be formulated and verified, with similar efficiency.

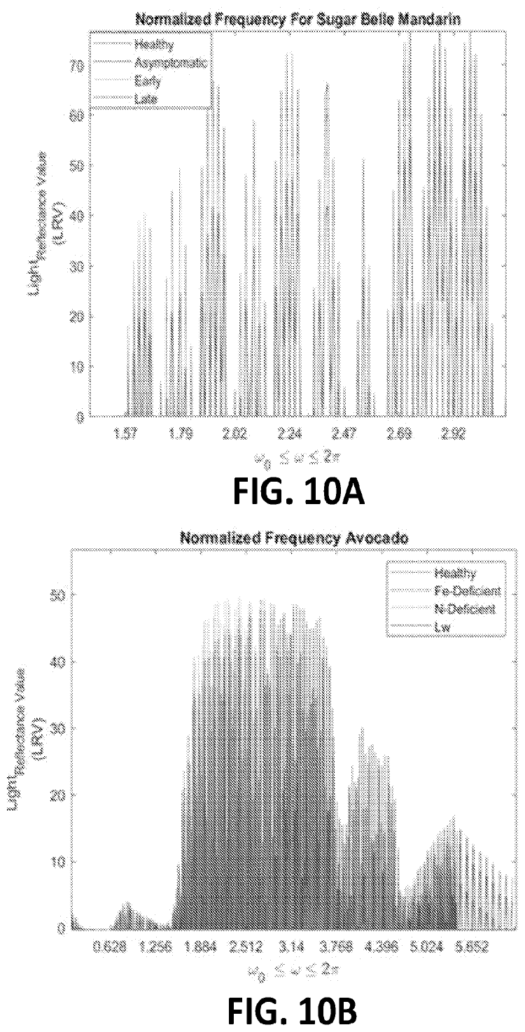

FIGS. 10A and 10B illustrate frequency data for the region of normalized frequency generated from reflectance signal data obtained from Sugar Belle Mandarin plants and avocado plants. In particular, FIG. 10A shows a normalized frequency graph of citrus (e.g., Sugar Belle Mandarin) plants, providing frequency data for Sugar Belle Mandarin plants with a healthy disease state, an asymptomatic disease state, an early stage disease state, and a late stage disease state. As shown, the magnitude of the frequency data for plants of each disease state are different, suggesting that disease states can be classified using the frequency data. Similarly, FIG. 10B shows a normalized frequency graph of avocado plants, including frequency data for avocado plants in a healthy disease state, an infected disease state for the disease of laurel wilt (“Lw”), an affected disease state for an iron deficiency, and an affected disease state for a nitrogen deficiency. As before, the magnitude of the frequency data for healthy plants, plants infected with laurel wilt, plants affected by iron deficiency, and plants affected by nitrogen deficiency are different, suggesting that different diseases and conditions (e.g., laurel wilt, iron deficiency, nitrogen deficiency) can be differentiated and classified using the frequency data. In both cases, it is shown that identifying the frequencies where regions of highest variance between different disease states occur allows for a reduced frequency representation of the signal to be used to identify the plant and the disease.

Thus, having the full-spectrum frequency data, various embodiments involve reducing the frequency data based at least in part on the selected one or more signal components, as previously described. The selected one or more signal components correlate to the feature frequencies of the frequency data that best describe the frequency data. That is, the selected one or more signal components are used in dimensional reduction in the Fourier domain. The greatest eigenvalue by spectral decomposition (e.g., in Equation 9) is significant in the sense that it relates to the component of greatest energy level of the reflectance signal data, through a transcendental function expansion. Equation 20 relates this to the cross-covariance matrix (e.g., Equation 7):

$$x ( y ) = {a}_{{λ}_{0}} {e}^{2 n j {f}_{{λ}_{0}} y + φ} + {a}_{{λ}_{1}} {e}^{2 π j {λ}_{1} y + φ} + {a}_{{λ}_{2}} {e}^{2 π {jf}_{{λ}_{2}} y + φ} + … {a}_{{λ}_{l}} {e}^{2 π j {f}_{{λ}_{ℓ}} y + φ} \qquad ( 20 )$$

Equations 21 and 22 constrain equation 20 to real and positive frequencies:

$$x ( y ) = \{ {\sum }_{i = 0}^{ℓ} {a}_{{λ}_{i}} {e}^{2 π {jf}_{{λ}_{i}} y + φ} \} \qquad ( 21 )$$

$$x ( y ) = {\sum }_{i = 0}^{ℓ} {a}_{{λ}_{i}} cos ( 2 π {f}_{{λ}_{i}} y + φ ) \qquad ( 22 )$$

In Equations 21 and 22, f_{λ}_{₁}, f_{λ}_{₂}, . . . , f_{λ}_{ᵢ}; represent the energy frequencies corresponding with the maximum/absolute value eigenvalues of the cross-covariance matrix, α_{λ}_{₁}, α_{λ}_{₂}, . . . , α_{λ}_{ᵢ} represent the Fourier coefficients at the corresponding energy frequencies, and φ represents the phase angle.

The inverse transformation of Equation 22 can then be used to appropriately truncate the frequency data to the feature frequencies that best describe the frequency data, as provided in Equations 23 and 24:

$$X ( f ) = \{ \frac{1}{l} {\sum }_{i = 0}^{ℓ} {a}_{{λ}_{i}} {e}^{- 2 π {jf}_{{λ}_{i}} y + φ} \} = \frac{1}{ℓ} {\sum }_{i = 0}^{ℓ} {a}_{{λ}_{i}} cos ( - 2 π {f}_{{λ}_{i}} y + φ ) \qquad ( 23 )$$

$$X ( f ) = \frac{1}{ℓ} {\sum }_{i = 0}^{ℓ} {a}_{{λ}_{i}} cos ( 2 π {f}_{{λ}_{i}} y + φ ) \qquad ( 24 )$$

Thus, this reduced-spectrum frequency data is associated with the eigenvalues of highest to lowest magnitude (f_{λ}_{₁}, f_{λ}_{₂} . . . f_{λ}_{ₗ}) of the cross-covariance matrix as found by the reduced modal approximation found in equation 12. Projection of the eigenvalues onto the Fourier domain space then results in mapping of the selected one or more signal components onto the frequency domain. Thus, the frequency data is truncated such that the frequencies associated with the highest signal energy are used to approximate the frequency data. Thus, by inverse transform, a morphological and statistical representation of the data for analysis purposes can then be obtained.

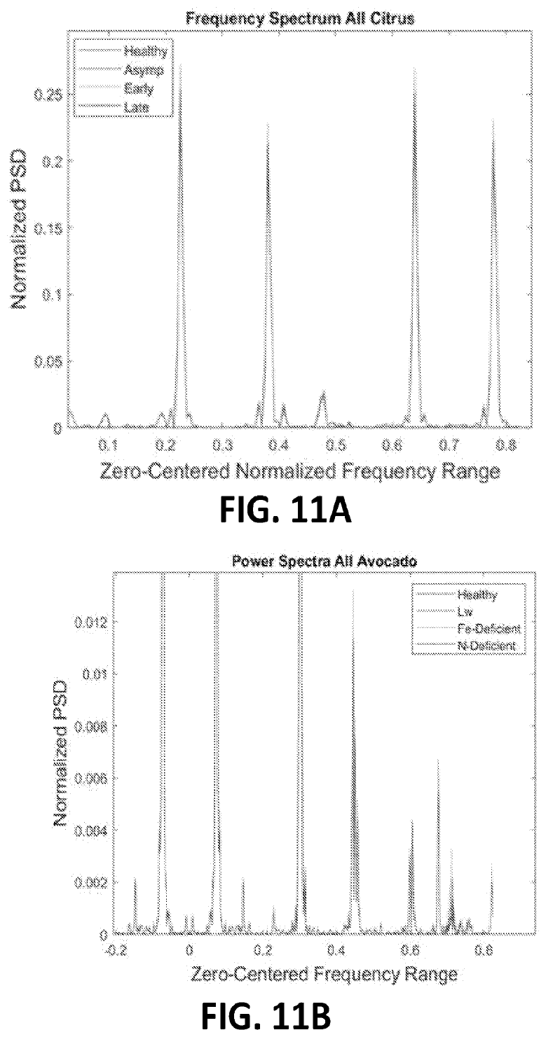

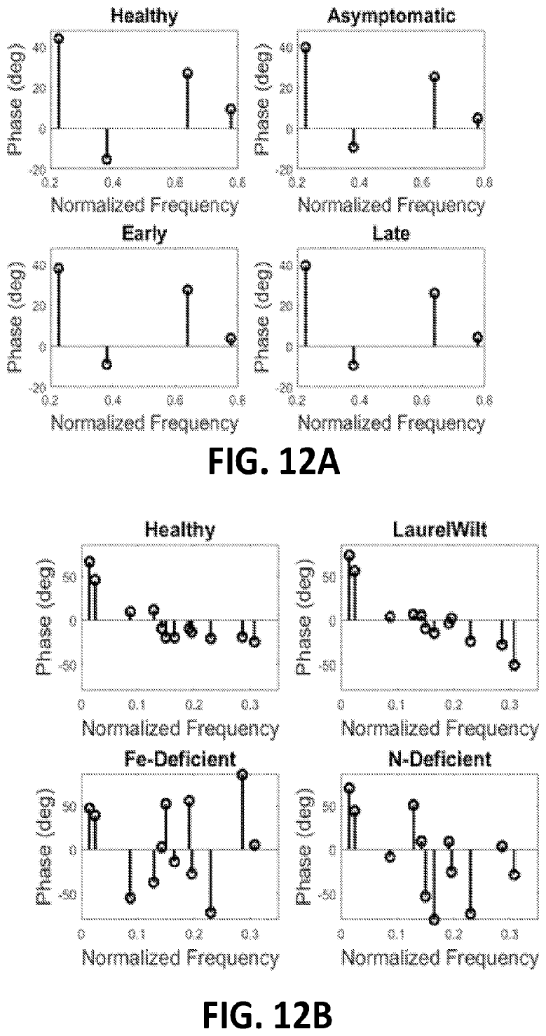

In general, the stochastic process of disease presents itself in both amplitude and phase in the frequency domain, as demonstrated in FIGS. 11A, 11B, 12A, and 12B.

FIG. 11A and FIG. 11B each illustrate example power density plots that show the frequencies associated with the highest signal energy.

FIG. 11A specifically illustrates an example power density plot for frequencies of reflectance signal data obtained from Sugar Belle Mandarin plants, including healthy citrus plants and citrus plants affected with canker (several stages). As shown, the example normalized modal frequencies of 0.226, 0.38, 0.6394, and 0.7773 are associated with the highest signal energy (e.g., between 0.21 and 0.263 normalized power spectral density). At the same time, the asymptomatic and early infected plant reflectance spectrum at the modal frequencies have higher magnitude, normalized power spectral densities than the healthy plants. The healthy plants register between 0.21-0.263 normalized PSD, and there are slight variations in power density between healthy and diseased plants. A summary of these magnitude differences is given in Table 1:

TABLE 1

| | |
|---|---|
| | Magnitude |
| Normalized | Normalized |
| Frequency | PSD |

| | | |
|---|---|---|
| 0.226 | 0.231 | 0.235 |
| | 0.263 | 0.254 |
| 0.38 | 0.212 | 0.210 |
| | 0.229 | 0.219 |
| 0.6394 | 0.227 | 0.233 |
| | 0.257 | 0.252 |
| 0.7773 | 0.223 | 0.221 |
| | 0.232 | 0.21 |

| | |
|---|---|
| H | A |
| E | L |

H—Healthy;
A—Asymptomatic;
E—Early;
L—Late

Similarly, FIG. 11B provides the KLE reduced Power density spectrum for healthy avocado, Lw affected avocado, and avocado plants with Fe, N deficiencies, thereby illustrating an example power density plot for frequencies of reflectance signal data obtained from avocado plants. As shown, various particular normalized modal frequencies are associated with high signal energy. Differences in amplitude for the various avocado groups show a significant reflectance energy difference for the infected vs. the healthy plant. The Fe-deficient has the highest PSD magnitudes, followed by Lw.

Accordingly, in some embodiments, the magnitude of signal energy at each normalized model frequency can be used to classify disease states and/or differentiate between diseases. In each power density plot illustrated by FIG. 11A and FIG. 11B, differences exist between plants of different disease states and different diseases. Accordingly, the magnitude of power spectral density can be applied as a parameter for classification of the plant due to the significant variations of this feature and used in generation of a reflectance signature for a plant and classification of a disease state for the plant.

In various embodiments, the reduced-spectrum frequency data includes phase information associated with each selected frequency (e.g., frequencies associated with the eigenvalues corresponding to the selected one or more signal components). FIG. 12A and FIG. 12B illustrate phase plots showing phase information associated with each selected frequency. In particular, applying the KLE, at a ROI 402-548 THz, and generating the phase spectrum for [symbol] =4,and [symbol] =14, respectively for citrus and avocado, produces the results reflected in the phase magnitude graphs shown in FIGS. 12A and 12B.

First, FIG. 12A provides the reduced phase domain signature for Sugar Belle mandarin plants, including healthy plants and plants affected with canker (several stages) after applying the KLE reduction technique, illustrating example phases for each of the four selected normalized modal frequencies of 0.226, 0.38, 0.6394, and 0.7773 for each disease state of healthy, asymptomatic, early stage, and late stage. Clearly, each disease state is associated with different phase characteristics across the selected normalized modal frequencies. For example, the first modal frequency has in the healthy spectrum a phase shift of 43.9° counter-clockwise compared to 38.1° to 39.6° in the infected species. The asymptomatic, early, and late citrus cancer development stages showed significant phase shift differences at the other modal frequencies. Table 2 below specifically provides the phases associated with each selected normalized modal frequency for each disease state which are illustrated in FIG. 12A:

TABLE 2

| | |
|---|---|
| Normalized | Phase |
| Frequency | (degrees) |

| | | |
|---|---|---|
| 0.226 | 43.9 | 39.64 |
| | 38.14 | 39.5 |
| 0.38 | −15.3 | −9.35 |
| | −9.06 | −9.55 |
| 0.6394 | 26.92 | 25.1 |
| | 27.48 | 25.88 |
| 0.7773 | 9.25 | 4.79 |
| | 3.74 | 4.26 |

| | |
|---|---|
| H | A |
| E | L |

H—Healthy;
A—Asymptomatic;
E—Early;
L—Late

Similarly, FIG. 12B shows the KLE-applied reduced phase spectrum for four distinct avocado groups ( [symbol] =14), illustrating example phases for various selected normalized modal frequencies for avocado plants of a healthy disease state, an infected disease state of laurel wilt, an affected disease state of iron deficiency, and an affected disease state of nitrogen deficiency. As shown, the phases for the selected normalized modal frequencies are significantly different between the different disease states and can therefore be used in differentiating between disease states and different diseases and conditions. For example, the first two modal frequencies have phases of approximately 74° to 41° for avocado plants of a healthy disease state, while the first two modal frequencies have phases of approximately 83° to 57° for avocado plants of the other disease states (e.g., laurel wilt, iron deficiency, nitrogen deficiency).

Accordingly, in some embodiments, phase differences are another parameter that can be used for differentiating healthy avocado from diseased and deficient plant reflectance signal data and used in generation of a reflectance signature for a plant and classification of a disease state for the plant.

Returning to FIG. 3, process 300 further comprises operation 308, which includes determining a reflectance signature for the plant using at least a portion of the reduced-spectrum frequency data. In various embodiments, the reflectance signature is unique for the plant and characterizes the disease state of the plant. In various embodiments, the reflectance signature is determined and generated from the reduced-spectrum frequency data.

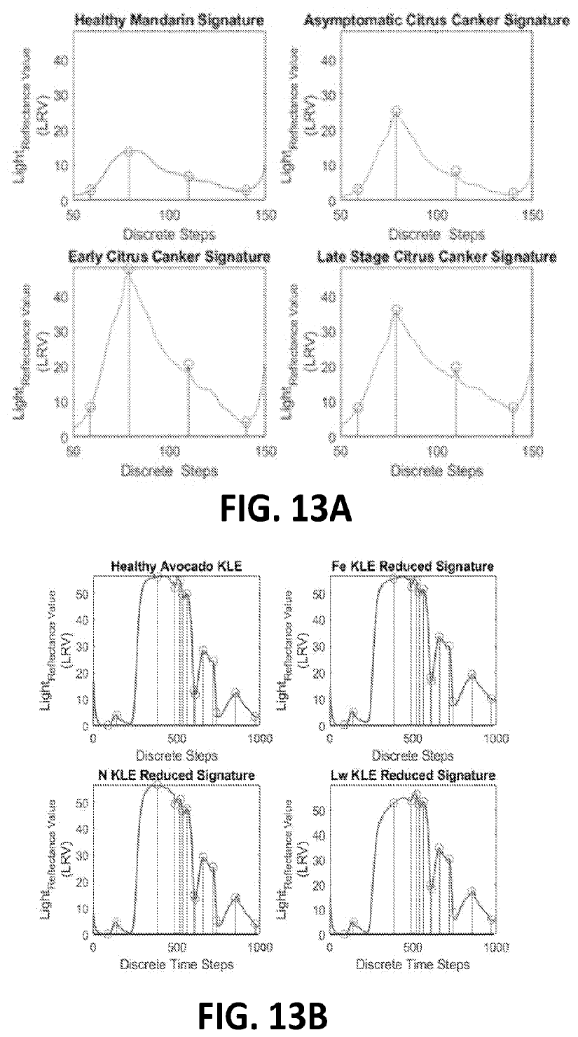

FIG. 13A and FIG. 13B illustrate example reflectance signatures for plants of different disease states. Specifically, FIG. 13A and FIG. 13B illustrate plots of Equation 24, or the truncated frequency data based at least in part on 1 number of frequencies.

More particularly, FIG. 13A shows an exemplary reflectance signature (e.g., KLE-reduced spatial domain signature) for healthy citrus plants and citrus plants affected with canker (several stages) by providing a rebuilt signal using the first four eigenvectors of greatest variance determined according to the methods previously discussed. In the illustrated example, the truncated frequency data is shown for a plant of a healthy disease state, a plant of an asymptomatic disease state, a plant of an early stage disease state, and a plant of a late stage disease state. FIG. 13A further indicates the four selected modal frequencies of 0.226, 0.38, 0.6394, and 0.7773 (e.g., 1 is four in the illustrated embodiment). As shown, the reflectance signatures are significantly different between plants of different disease states. Morphological effects of the disease change the spectrum in the 50-150 discrete sampled steps region. The envelope becomes peaky as soon as disease is present in the leaf. It is also important to note that at discrete step 109, the diseased leaves show a perceptible, increased magnitude spectral component in the reflectance spectrum.

Similarly, FIG. 13B illustrates reflectance signatures for avocado plants of a healthy disease state, an infected disease state of laurel wilt, an affected disease state of iron deficiency, and an affected disease state of nitrogen deficiency. Specifically, FIG. 13B illustrates the truncated frequency data according the Equation 24 for avocado plants of such disease states. In the illustrated example, the regions from 650-900 discrete steps show enough variation in signature to be able to identify the discrepancy caused by Lw disease and nutritional deficiency. The data are similar but variations with the infected plant show higher amplitudes and morphological changes to the data that are distinguishable using standard statistical methods.

Thus, as described above, the reduced-spectrum frequency data may include the truncated frequency series described by Equation 24 and illustrated in FIG. 13A and FIG. 13B, power spectral density magnitudes for each selected frequency as illustrated in FIG. 11A and FIG. 11B, and phases for each selected frequency as illustrated in FIG. 12A and FIG. 12B. In various embodiments, the reflectance signature then comprises at least one of the truncated frequency series, power spectral density magnitudes for each selected frequency, or phases for each selected frequency.

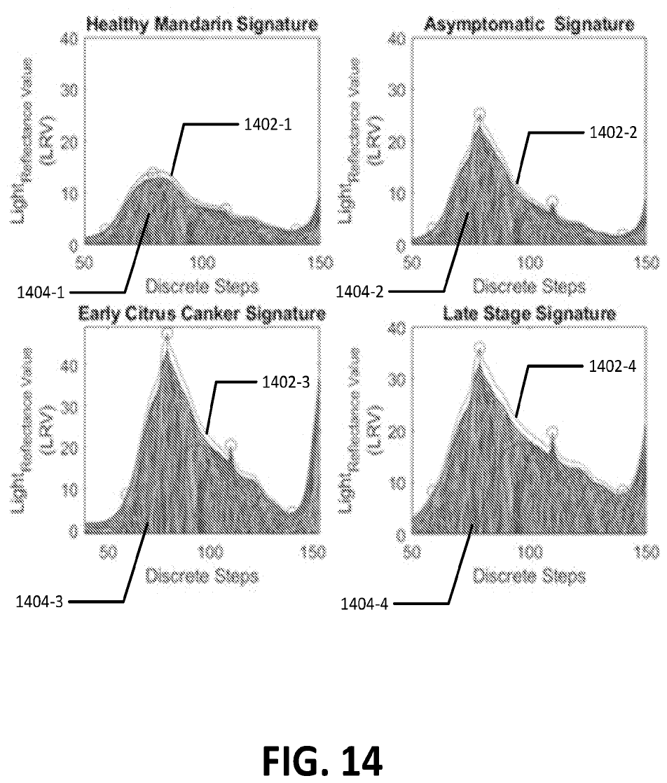

FIG. 14 shows exemplary reflectance signatures 1402 (e.g., KLE-reduced spatial domain signature) based at least in part on reduced frequency data overlaid upon inverse transforms of non-reduced reflectance signal data 1404 for citrus plants in the healthy, asymptomatic, early stage, and late stage disease states. As previously discussed, the equations for the reduced KLE signatures 1402 are found by implementing Equation 24, with the first four highest eigenvalue frequencies (normalized) for the citrus data. On the other hand, the non-reduced signature 1404 is generated via an inverse transform of the data in the complete spectrum from 302.8 THz to 908 THz. It is important to note that the accuracy of the spectra is not compromised by the reduced frequency set.

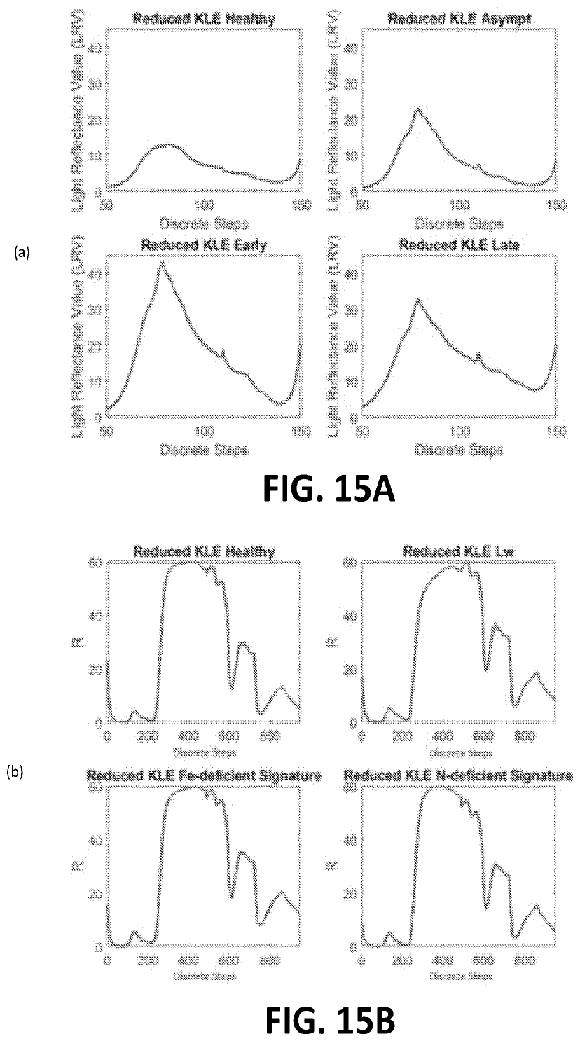

FIGS. 15A and 15B show signal envelopes for the KLE-reduced signatures (e.g., as shown in FIGS. 13A and 13B). More particularly, FIG. 15A shows signal envelopes for citrus plants at each disease state, while FIG. 15B shows signal envelopes for avocado plants at each disease state. These signal envelopes represent averaged signatures that can be used as representative biomarkers for plant reflectance signatures for their respective plant species and/or disease states. Variations are shown in the signatures for citrus cancer infected plants, and avocado infected with Lw and other deficiencies. Here, when classifying the disease states of plants, various regions of interest (ROIs) can be used for pattern differencing or feature extraction regions, depending on what type of detection or classification is being applied.

In general, spatial signature envelopes for the overall average of each plant group (e.g., healthy citrus, asymptomatic citrus, early stage diseased citrus, late stage diseased citrus, healthy avocado, Lw affected avocado, Fe-deficient avocado, N-deficient avocado) are generated by Inverse Fourier Transform by using the KLE reduced frequencies, as previously described. This averaged signature formation method from the KLE and FT allows for estimating a signature database for each plant group within each species. Then, methods for correlation, matched filtering, principle component analysis or other statistical and artificial intelligence methods can be applied for classification of plant diseases and disease development states according to spectral signature analysis. By using the KLE-FT method and separating each signature in spatial domain according to Mercer modes, a method to categorize the plants by their signature can be used for classification.

Accordingly, returning to FIG. 3, upon generation of the reflectance signature for the plant (e.g., at least one of the truncated frequency series, power spectral density magnitudes, or phases), various actions can be performed, and in the illustrated embodiment, at least one of operation 310A and operation 310B are performed. Referring first to operation 310A, a signature database (e.g., storage system 165) is generated and/or updated with the reflectance signature for the plant. In various embodiments, the signature database is configured to store an average reflectance signature for plants of the same disease state. Additionally or alternatively, the signature database is configured to store individual reflectance signatures for different plants. In various embodiments, the disease classification system 180 is configured to retrieve and/or access reflectance signatures (e.g., average, individual) stored by the signature database to use in classification of disease state.

In this regard, operation 310B comprises classifying a disease state of the plant based at least in part on the reflectance signature for the plant. In various embodiments, the disease classification system 180 is configured to use one or more machine learning models to determine a disease state classification for the plant based at least in part on providing the reflectance signature (e.g., at least one of the truncated frequency series, power spectral density magnitudes, or phases) as input to the one or more machine learning models. In various embodiments, the one or more machine learning models are configured (e.g., trained) using supervised learning techniques and/or semi-supervised learning techniques using historical reflectance signatures stored by the signature database. In various embodiments, the disease classification system 180 is configured to store a reflectance signature assigned with a predicted and inferred disease state classification in the signature database.

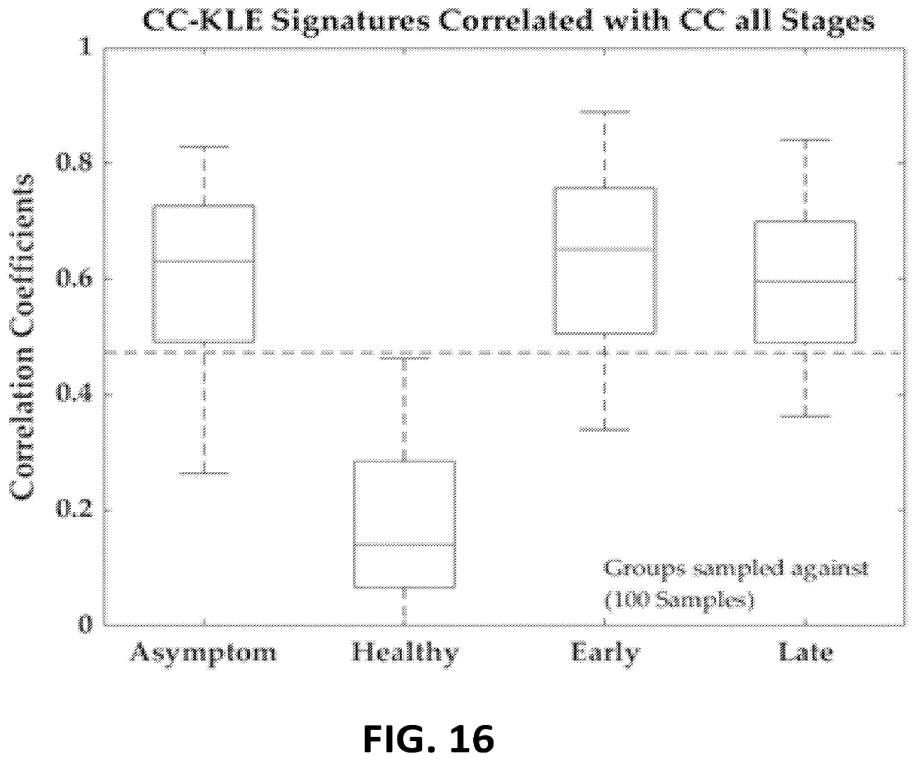

In various embodiments, the one or more machine learning models comprise a correlation classification model. FIG. 16 illustrates bivariance correlation coefficients for Sugar Belle Mandarin plants of various disease states, specifically healthy, asymptomatic, early, and late. Randomized reflectance signatures and correlated against the reflectance signatures determined and generated according to at least operation 308, and as shown in FIG. 13A, the correlation coefficients associated with plants of the healthy disease state are significantly different than those associated with plants of other disease states. Here, the reflectance signatures generated based at least in part on the reduced frequency data reflect enough morphological disparities that a formal bivariate correlation such as that illustrated in FIG. 16 produces correct classification results for diseased versus non-diseased plants. Accordingly, a disease state may be classified using bivariance correlation techniques and a classification model.

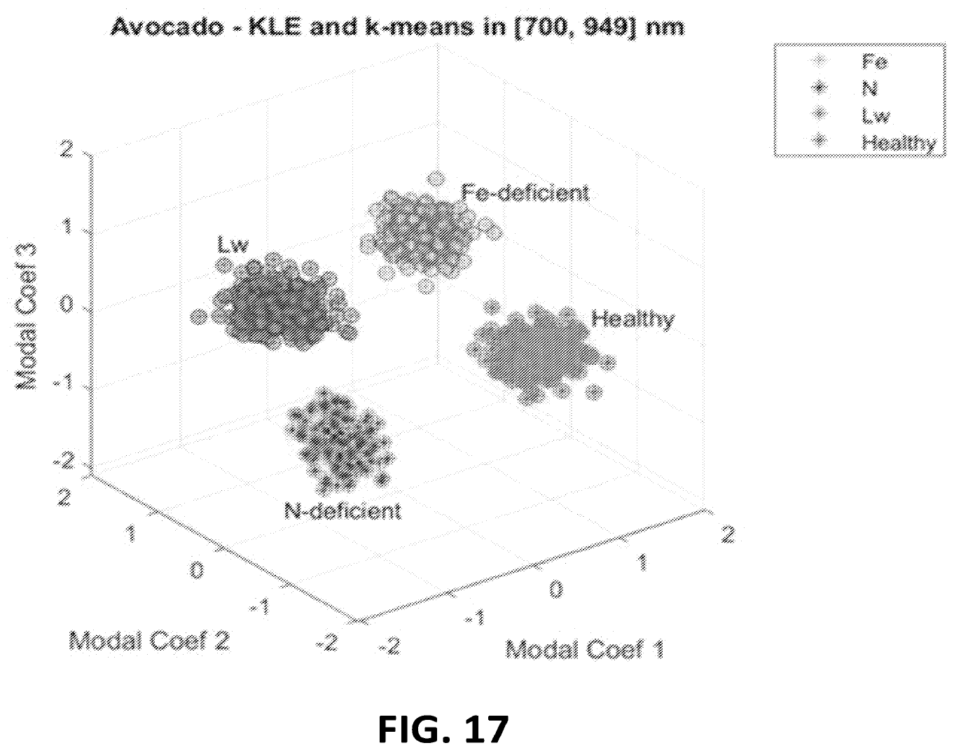

In various embodiments, the one or more machine learning models comprise a clustering model, such as a k-means model, and classification of disease state is performed using such a clustering model. FIG. 17 illustrates a k-means characterization of avocado plants of different disease states, specifically healthy, infected with laurel wilt, affected with iron deficiency, and infected with nitrogen deficiency, for three example selected modal frequencies. As shown, plants of each disease state are clustered together, and each cluster can be easily classified. More particularly, in the illustrated example, the KLE is applied to the avocado reflectance signal data and the number of primary Mercer modal components necessary for representing over 47% of the data was obtained to be [symbol] =3, which accounts for the three-dimensional characterization of the four avocado signatures illustrated in FIG. 17. In this case, using the first three eigenvalues results in 100% classification accuracy using the previously described clustering model. Accordingly, classification of disease state may be performed using clustering.

Returning to FIG. 3, subsequent to classification of a disease state of the plant, various automated state-based actions may be performed using the classified disease state of the plant, as indicated in step 312. For example, the disease state of the plant may be visualized in a map using one or more indicators associated with each disease state. Remedial actions for plants classified as being in an infected disease state or the like may also be performed, such as automatically causing water, nutrients, and/or the like to be provided to the plant via actuators within the area of interest 130 connected to the disease classification system 180 via the network 145, the actuators being configured to perform the respective remedial actions with respect to individual plants or groups of plants within the area of interest 130. Classification of disease states may be repeatedly performed for a plurality of plants in the area of interest 130, and a disease state map may be generated for the area of interest 130. In various embodiments, a notification or alert indicating the classified disease state and/or location information for the plants in question is provided to the user device 125.

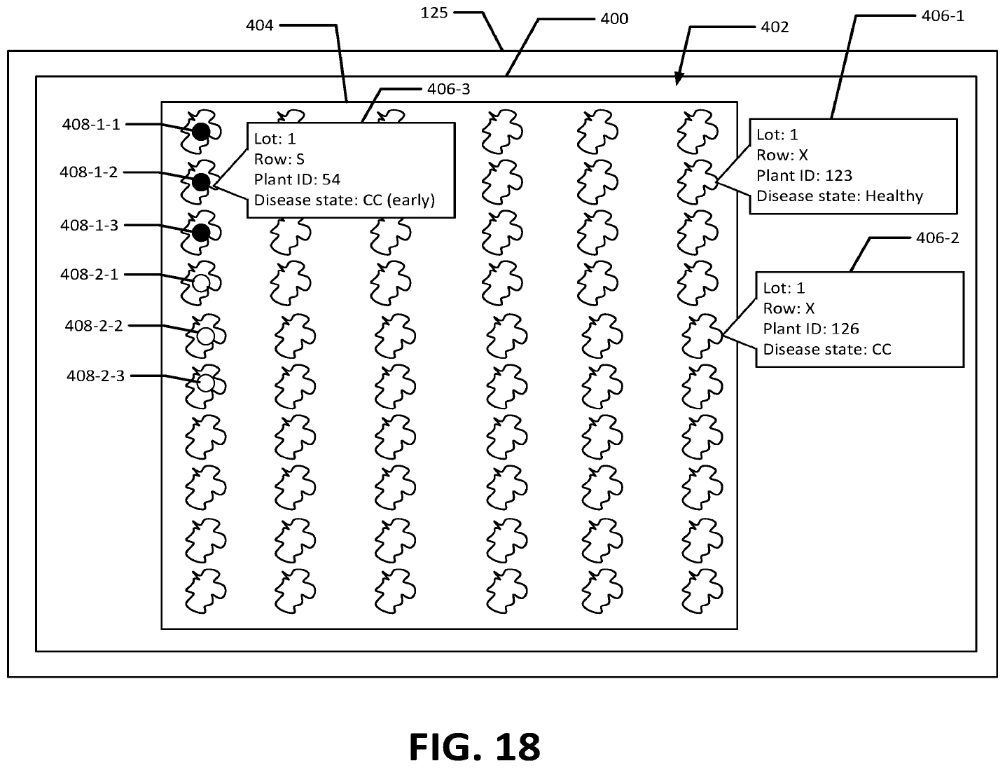

FIG. 18 shows an exemplary graphical user interface (GUI) 404 rendered on a display 400 of one or more user devices 125. In general, the GUI 404 presents information about the disease state of various plants based at least in part on classification results from the disease classification system 180 and/or information stored in storage 165. The GUI 404 includes various screens or views for displaying information and receiving input from a user (e.g., by detecting contact between the user and a touchscreen display in certain regions of the display). Additionally, the GUI 404 comprises various graphical elements (e.g., icons, virtual buttons, menus, textual information, information panes, images).

More particularly, the GUI 404 comprises a map pane 404, which presents a map or graphical representation of a particular area of interest 130 being presented to the user. In various embodiments, the graphical representation is an image captured from over the area of interest 130 (e.g., satellite image). In the illustrated example, the map pane 404 displays a disease state map showing multiple rows of plants.

In general, various graphical elements are overlaid on the disease state map in different positions based upon positions of graphical representations of the various plants depicted in the picture to which the graphical elements pertain (e.g., based at least in part on location, position, and/or orientation data included in reflectance signal data gathered for the plant).

For example, the map pane 404 includes detail panes 406, which provide textual information about selected plants and are positioned on the GUI 404 with respect to the disease state map in a manner that indicates the plant about which the information is being presented, in this case by being displayed adjacent to and pointing to the respective depictions of the plants on the disease state map. In one embodiment, the detail panes 406 for plants are displayed in response to user selection of graphical representations of the plants within the disease state map or any graphical element representing the plant in question. In the illustrated example, there are three detail panes 406. Detail pane 406-1 points to one particular depiction of a plant in the disease state map and provides textual information indicating that the plant in question is non-diseased or “healthy.” Detail pane 406-2 points to a different depiction of a plant in the disease state map and provides textual information indicating that the plant in question is diseased, namely that it is infected with citrus canker. Detail pane 406-3 points to a still another depiction of a plant in the disease state map and provides textual information indicating that the plant in question is diseased, namely that it is infected with citrus canker in the early developmental stage.

In another example, disease state indicators 408 are overlaid on top of the depictions of the respective plants on the disease state map and have visual characteristics based at least in part on which disease states are being indicated. In the illustrated example, there are disease indicators 408-1 and healthy indicators 408-2, with each type of indicator having a different color, shade, and/or hue. The disease indicators 408-1 are overlaid on the disease state map over the graphical representations of plants that were determined to be diseased, while the healthy indicators 408-2 are overlaid on the disease state map over the graphical representations of plants that were determined to be healthy. For the sake of clarity, only three of each type of indicator is shown. However, in various embodiments, each plant depicted in the disease state map

Thus, various embodiments provide various technical advantages in disease detection and classification for plants. By examining reflectance signal data, inherent biomarkers in plants are examined, thereby providing a deeper and more accurate analysis compared to visual analysis. Such examination using reflectance signal data enables earlier detection and classification of disease, as visual manifestation of symptoms for various diseases may only occur in later disease stages. Further, various embodiments involve reduction of reflectance signal data to high-energy and high-variance frequency components that carry the majority of information of the reflectance signal data. This reduction enables improved efficiency in computational and processing resources, due to there being less data and fewer dimensions thereof to analyze.

### V. Computer Program Products

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described above with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### VI. Conclusion

It should be understood that the examples and embodiments described herein are for illustrative purposes only and that various modifications or changes in light thereof will be suggested to persons skilled in the art and are to be included within the spirit and purview of this application. Although the present disclosure is considered complete and comprehensive, additional context and insight may be gleaned from the appendices attached alongside this specification (which describes generally systems, apparatuses, and methods in accordance with embodiments herein).

Many modifications and other embodiments of the present disclosure set forth herein will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claim concepts. Although specific terms are employed herein, they are used in a generic and descriptive sense only and not for purposes of limitation.

1. A method for detecting diseases in one or more plants, the method comprising:
receiving reflectance signal data for a plant;
identifying a plurality of signal components of the reflectance signal data;
selecting signal components from the plurality of signal components, the selected signal components having a variance satisfying a variance threshold, wherein the variance is between particular signal components of reflectance signal data for a non-diseased plant of a plant species and corresponding signal components of reflectance signal data for a diseased plant of the plant species;
generating reduced-spectrum frequency data based at least in part on the reflectance signal data and the selected signal components;
generating a reflectance signature for the plant based at least in part on the reduced-spectrum frequency data; and
determining a disease state of the plant based at least in part on the reflectance signature.

2. The method of claim 1, further comprising generating a signature database configured to describe one or more average reflectance signatures for a plant species associated with the plant based at least in part on the reflectance signature for the plant, each of the one or more average reflectance signatures being associated with a particular disease state of the plant species.

3. The method of claim 2, wherein the one or more average reflectance signatures include healthy plant signatures, asymptomatic plant signatures, early disease stage plant signatures, late disease stage plant signatures, and/or nutrient-deficient plant signatures.

4. The method of claim 1, wherein the reflectance signature comprises at least one of a truncated frequency series associated with the reflectance signal data, one or more power spectral density magnitudes associated with the selected signal components, or one or more phases associated with the selected signal components.

5. The method of claim 1, wherein the selected signal components are identified based at least in part on using Karhunen-Loeve Expansion as a kernel function for Mercer's Theorem.

6. The method of claim 1, wherein the disease state of the plant is determined based at least in part on providing the reflectance signature to one or more machine learning models configured to predict the disease state of a plant when provided with an input reflectance signature.

7. The method of claim 6, wherein the one or more machine learning models comprise a clustering model, a bivariance correlation model, and/or a classification model.

8. The method of claim 1, further comprising performing one or more automated state-based actions according to the disease state of the plant.

9. The method of claim 8, wherein the one or more automated state-based actions comprises updating an area-of-interest map to indicate the disease state of the plant positioned in an area-of-interest and presenting the area-of-interest map via displays of one or more user devices.

10. The method of claim 9, wherein the reflectance signal data for the plant was collected via a sensing platform comprising one or more data collection devices configured to acquire reflectance signal data of the plant within an area of interest, the one or more data collection devices being configured to record position and orientation data associated with the collected reflectance signal data.

11. A system for detecting diseases in one or more plants by:
receiving reflectance signal data for a plant, identifying a plurality of signal components of the reflectance signal data;
selecting, from the plurality of signal components, signal components having a variance satisfying a variance threshold;
generating reduced-spectrum frequency data based at least in part on the reflectance signal data and the selected signal components;
generating a reflectance signature for the plant based at least in part on the reduced-spectrum frequency data; and
determining a disease state of the plant based at least in part on the reflectance signature, wherein the variance is between particular signal components of reflectance signal data for a non-diseased plant and corresponding signal components of reflectance signal data for a diseased plant.

12. The system of claim 11, further comprising a signature database configured to describe one or more average reflectance signatures for a plant species associated with the plant based at least in part on the reflectance signature for the plant, each of the one or more average reflectance signatures being associated with a particular disease state of the plant species.

13. The system of claim 12, wherein the one or more average reflectance signatures include healthy plant signatures, asymptomatic plant signatures, early disease stage plant signatures, late disease stage plant signatures, and/or nutrient-deficient plant signatures.

14. The system of claim 12, wherein the one or more average reflectance signatures comprise at least one of a truncated frequency series associated with the reflectance signal data, one or more power spectral density magnitudes associated with the selected signal components, or one or more phases associated with the selected signal components.

15. The system of claim 11, wherein the selected signal components are identified based at least in part on using Karhunen-Loeve Expansion as a kernel function for Mercer's Theorem.

16. The system of claim 11, wherein the system determines the disease state of the plant based at least in part on providing the reflectance signature to one or more machine learning models configured to predict the disease state of a plant when provided with an input reflectance signature.

17. The system of claim 16, wherein the one or more machine learning models comprise a clustering model, a bivariance correlation model, and/or a classification model.

18. The system of claim 11, wherein the system performs one or more automated state-based actions according to the disease state of the plant.

19. The system of claim 18, wherein the one or more automated state-based actions comprises updating an area-of-interest map to indicate the disease state of the plant positioned in an area-of-interest and presenting the area-of-interest map via displays of one or more user devices.

20. The system of claim 19, wherein the reflectance signal data for the plant was collected via a sensing platform comprising one or more data collection devices configured to acquire reflectance signal data of the plant within an area of interest, the one or more data collection devices being configured to record position and orientation data associated with the collected reflectance signal data.

21. A computer program product for detecting diseases in one or more plants, the computer program product comprising at least one non-transitory computer-readable storage medium having computer-readable program code portions stored therein, the computer-readable program code portions configured to:
receive reflectance signal data for a plant;
identify a plurality of signal components of the reflectance signal data;
select signal components from the plurality of signal components, the selected signal components having a variance satisfying a variance threshold, wherein the variance is between particular signal components of reflectance signal data for a non-diseased plant of a plant species and corresponding signal components of reflectance signal data for a diseased plant of the plant species;
generate reduced-spectrum frequency data based at least in part on the reflectance signal data and the selected signal components;
generate a reflectance signature for the plant based at least in part on the reduced-spectrum frequency data; and
an executable portion configured to determine a disease state of the plant based at least in part on the reflectance signature.
